SEQUENCE LISTING (17 sequences)

SEQ ID NO: 1 - protein, 25 aa, synthetic construct
AGTEKIFQRLKKTIQEGKKIAKRWW

SEQ ID NO: 2 - protein, 16 aa, synthetic construct
SAVWRHWRRFWLRKRK

SEQ ID NO: 3 - protein, 25 aa, synthetic construct
AWKKTIRQYLKNKIKKKGRKAVIAW

SEQ ID NO: 4 - protein, 18 aa, Rana chensinensis
SAVGRHGRRFGLRKHRKH

SEQ ID NO: 5 - protein, 18 aa, synthetic construct
SAVWRHWRRFWLRKHRKH

SEQ ID NO: 6 - protein, 16 aa, synthetic construct
SAVWRHWRRFWLRKHR

SEQ ID NO: 7 - protein, 25 aa, Bacillus safensis
AGKETIRQYLKNEIKKKGRKAVIAW

SEQ ID NO: 8 - protein, 25 aa, synthetic construct
AWKKTIRQYLKNKIKKKGRKAVIAW

SEQ ID NO: 9 - protein, 25 aa, synthetic construct
AGKETIRQFLKKKIQEKGKRATIAW

SEQ ID NO: 10 - protein, 25 aa, synthetic construct
AGTEKIFQRLKKTIQEGKKIAKRWW

SEQ ID NO: 11 - protein, 26 aa, synthetic construct
CAWKKTIRQYLKNKIKKKWRKAVIAW

SEQ ID NO: 12 - protein, 26 aa, synthetic construct
CAWKEKIRKKLKNEIKKKWRKAVIAW

SEQ ID NO: 13 - protein, 21 aa, synthetic construct
FTMKKSLLLLFFLGTINFSLC

SEQ ID NO: 14 - protein, 16 aa, synthetic construct
CSAVWRWRRFWLRKRK

SEQ ID NO: 15 - protein, 17 aa, synthetic construct
CKGLKKLLKLLKKLLKL

SEQ ID NO: 16 - protein, 16 aa, synthetic construct
CSAVWRWRRFWLRKRK

SEQ ID NO: 17 - protein, 15 aa, synthetic construct
CLKKLLKKLKKLLKK

(19) United States
(12) Patent Application Publication
(10) Pub. No.: US 2026/0355345 A1
(43) Pub. Date: Nov. 12, 2026

(54) PEPTIDE-CONJUGATED PHAGE-MIMICKING NANOPARTICLES FOR INFLAMMATION FREE WOUND HEALING

(71) Applicant: UNIVERSITY OF NOTRE DAME DU LAC, South Bend, IN (US)

(72) Inventor: Prakash Daniel NALLATHAMBY, South Bend, IN (US)

(21) Appl. No.: 19/487,191

(22) PCT Filed: May 28, 2024
(86) PCT No.: PCT/US2024/031264

§ 371 Date: Nov. 24, 2025

Related U.S. Application Data
(60) Provisional application No. 63/504,503, filed on May 26, 2023.

(51) Int. Cl. A61K 47/69; A61K 9/00; A61K 38/00; A61P 31/04; B82Y 5/00; C07K 14/00

(52) U.S. Cl. CPC A61K 47/6923; A61K 9/0014; A61P 31/04; C07K 14/001; A61K 38/00; B82Y 5/00

(57) ABSTRACT

An antibacterial nanoparticle comprising an inner silica core; a gold (Au) nanosphere surrounding the inner silica core; a surface layer comprising silver (Ag) surrounding the gold nanosphere or the silver is alloyed into the gold nanosphere; and an antimicrobial peptide conjugated to the surface layer, wherein the antimicrobial peptide is selected from the group consisting of a cysteine terminated Syn71 peptide, a cysteine terminated MC1-2 peptide, and a cysteine terminated Syn20 peptide.

### RELATED APPLICATIONS

This application claims priority under 35 U.S.C. § 119 (e) to U.S. Provisional Patent Application No. 63/504,503, filed May 26, 2023, which is incorporated herein by reference.

### BACKGROUND OF THE INVENTION

The main treatment method for decades against bacterial infections has been the use of antibiotics. The irresponsible, widespread use of antibiotics, however, has paved the way for a rapid increase in the development of antibiotic resistance in bacteria. The increase in resistance coupled with the lack of new antibiotics due to the development slowdown in the 1990s has made many of the previously easily treatable bacterial infections more challenging to tackle. The spread of antibiotic resistance and the resulting emergence of multidrug-resistant bacteria has become one of the main threats to the public health system. Especially problematic are the emerging hospital-acquired infections (HAIs) caused by a group of pathogens called ESKAPE, including Enterococcus faecium, Staphylococcus aureus, Klebsiella pneumoniae, Acinetobacter baumannii, Pseudomonas aeruginosa, and Enterobacter spp. These bacteria exhibit multidrug resistance to most of the antibiotics available and cause a variety of infections, such as pneumonia, skin and bloodstream infections. To fight these multidrug-resistant bacteria infections, researchers have turned their focus on the development of alternative methods.

Nanoparticle-based antibacterial systems have become popular in the fight against antibiotic-resistant bacteria. Metal nanoparticles have been extensively studied and display a promising potential as alternatives to conventional antibiotics due to their high antibacterial activity. Metal nanoparticles act via various mechanisms of action, such as the generation of reactive oxygen species (ROS), the release of cations, interactions with bacterial membrane and biomolecular damages. Heavy metal nanoparticles, such as silver nanoparticles, generate ROS with high affinity to thiol (—SH) groups such as cysteine. Silver and its antibacterial effect have been a widely researched area due to its long history of usage in treating infections and silver nanoparticles being the most effective against bacteria while being more biocompatible than other metal nanoparticles. The unique set of physicochemical properties of silver, such as size distribution, agglomeration, surface chemistry and ion release, are the key factors for the antibacterial activity of silver nanoparticles. To enhance the overall antibacterial effect, some researchers have started investigating the combinations of various metals to enhance the antibacterial effect and lower the cytotoxicity to mammalian cells, while others have focused on the synergistic antibacterial effect exhibited by the nanoparticles and antimicrobial peptides (AMPs).

AMPs are an important novel source of antibacterial compounds used as an alternative to conventional antibiotics. These peptides are usually positively charged short peptides existing as part of the innate immune system in humans, animals and plants. AMPs exhibit a wide antibacterial spectrum with complex mechanisms induced by the direct interactions of the peptides with bacterial membranes, leading to membrane disruption, or entry into the cell and interactions with intracellular components. Acting through various mechanisms reduces the possibility of bacteria developing resistance towards AMPs to very low.

At current antibiotics discovery and development rates, we will eventually lose to antibiotic-resistant strains. By 2050, antibiotics-resistant strains of bacteria will kill more patients per year than all cancers combined. Therefore, there is a desperate need to look for methods beyond antibiotics to kill harmful bacteria and slow the rise of drug-resistant bacterial pathogens. The present disclosure satisfies these needs.

### SUMMARY OF THE INVENTION

The development of antibiotic resistance and the resulting emergence of multidrug-resistant bacteria has become one of the main threats in the public health system, commonly leading to nosocomial infections. Many researchers have turned their focus to developing an alternative class of antibacterial systems based on various nanomaterials. We have developed an antibiotic-free nanoparticle system, inspired by naturally occurring bacteriophages to fight antibiotic resistant bacteria. Our developed phage-mimicking nanoparticles (PhaNPs) display structural mimicry of protein-turret distribution on the head structure of bacteriophages. By mimicking phages, we are able to take advantage of their evolutionary constant shape and their high antibacterial activity while avoiding the immune reaction of the human body, potentially caused by real phages.

The present disclosure describes phage-mimicking nanoparticles (PhaNPs) combined with various synthetic antimicrobial peptides. The design of PhaNPs were based on the structure of tailless bacteriophages. It is assembled hierarchically, first synthesizing the silica core nanoparticle, followed by deposition of gold nanospheres onto the silica core, and finally coating the gold nanospheres on the silica core with a silver layer. By taking advantage of this hierarchical assembly, we were able to exhibit high growth inhibition of several clinically relevant ESKAPE pathogens (up to 100%), while minimizing the cytotoxicity to mammalian cells. Here, we hypothesize that by adding another hierarchical assembly unit in the form of AMPs conjugated onto the PhaNPs (PhaNP@Peptide), we are able to further increase the antibacterial activity and the specificity towards bacteria cells. We carried out a series of growth curve measurements on five Gram-positive and Gram-negative bacteria strains (with four ESKAPE pathogens) in the presence of different concentrations of PhaNP@Peptide. We tested two different sizes of PhaNP@Peptide (20 nm and 95 nm), and three PhaNP@Peptide variations conjugated with different AMPs (MC1-2, Syn20, and Syn71) to study the dependence of the antibacterial activity on the nanoparticle size and the peptide used. We are able to show that the new PhaNP@Peptide design has a high antibacterial activity towards bacteria, irrespective of the Gram group. It was shown that the antibacterial activity is size dependent, with a bigger nanoparticle design (95 nm) exhibiting a stronger antibacterial effect.

Accordingly, the disclosure provides an antibacterial nanoparticle comprising: an inner silica core; a gold (Au) nanosphere surrounding the inner silica core; a surface layer comprising silver (Ag) surrounding the gold nanosphere or the silver is alloyed into the gold nanosphere; and an antimicrobial peptide conjugated or chemisorbed to the surface layer, wherein the antimicrobial peptide is selected from the group consisting of a cysteine terminated Syn71 peptide, a cysteine terminated MC1-2 peptide, and a cysteine terminated Syn20 peptide.

In some embodiments, the antibacterial peptide is the cysteine terminated Syn71 peptide having an amino acid sequence of CAGTEKIFQRLKKTIQEGKKIAKRWW (SEQ ID NO: 1), or the antibacterial peptide is the cysteine terminated MC1-2 peptide having an amino acid sequence of CSAVWRHWRRFWLRKRK (SEQ ID NO: 2), or the antibacterial peptide is the cysteine terminated Syn20 peptide having an amino acid sequence of CAWKKTIRQYLKNKIKKKGRKAVIAW (SEQ ID NO: 3).

In some embodiments, the inner silica core has a silanized outer surface. In some embodiments, the nanoparticle has an average diameter of about 15 nm to about 30 nm. In some embodiments, the nanoparticle has an average diameter of about 45 nm to about 70 nm or about 90 nm to about 100 nm.

In other embodiments, a Syn71 peptide is conjugated to the silver surface layer of the antibacterial nanoparticle at an average concentration of about 40 μM to about 60 μM, or the MC1-2 peptide is conjugated to the surface layer at an average concentration of about 40 μM to about 60 μM, or the Syn20 peptide is conjugated to the surface layer at an average concentration of about 40 μM to about 60 μM.

The disclosure also provides for antibacterial compositions comprising an antibacterial nanoparticle as described herein and pharmaceutically acceptable diluent, excipient, or carrier. In some embodiments, the composition comprises a population of the antibacterial nanoparticle having an average of from about 1 μM to about 150 μM of the antimicrobial peptide conjugated to the surface layer. In some embodiments, the antibacterial composition is formulated as a topical cream or ointment.

Also provide are methods of treating a bacterial infection in a subject having a bacterial infection comprising administering an effective amount of the antibacterial nanoparticles according to claim 1 to the subject, wherein the antibacterial nanoparticles reduce growth of the bacterial infection by greater than about 95%, kills bacteria of the bacterial infection, or both, thereby treating the bacterial infection. The antibacterial nanoparticle or a composition comprising the antibacterial nanoparticles is administered topically or parenterally.

In some embodiments, a medical product may comprise the antibacterial particles or compositions comprising the antibacterial nanoparticles. For example, the medical product may be one or more of a medical implant, a dental implant, and a surgical instrument, and optionally, the antibacterial nanoparticle (or composition thereof) is disposed on a surface of the medical implant, the dental implant, and the surgical instrument.

In other embodiments, the disclosure provides for a medical bandage comprising the antibacterial nanoparticle the composition there of, where the medical bandage may be applied to the wound to facilitate would healing.

Advantageously, embodiments of the invention provide for 1) immediate wound stabilization; 2) anti-inflammatory; 3) gentle wound healing; and 4) multiple formulations that can be formulated to be topical or can be formulated to be delivered intravenously.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-B. A visual representation of (A) the PhaNP synthesis process, (B) growth curve measurement preparation.

FIG. 2A-C. Nanoparticle characterization. (A) left) TEM image of SiNPs before surface modifications and right) the size distribution of SiNPs showing an average diameter of 20.47 nm+2.14 nm. (B) left) TEM image of PhaNPs used for Syn71 conjugation and right) the size distribution of PhaNPs showing an average diameter of 20.18 nm+2.41 nm. (C) The energy-dispersive X-ray spectrum (EDXS) of PhaNPs confirmed their elemental composition. Distinct energy peaks for Si(Kα=1740 eV), O (Kα=525 eV), Au (Mα=2123 eV, Lα=9.713 keV) and Ag (Kα=22.163 keV, Lα=2.983 keV) were detected. Al peak is present due to SEM stub material; Mg, Na and Cl peaks are due to the solvent used (PBS).

FIG. 3A-F. (A) Biocompatibility of PhANP@Syn71 on the HaCaT human keratinocyte cell line. The average of four replicates for each sample was computed together with standard deviations. (B) SEM images of HaCaT cells incubated with PhaNP@Syn71. (C)-(F) In vitro Scratch Closure Assay on HaCaT cells showed no cell migration in the presence of PhaNP@Syn71, while Syn71 peptide only presence allowed cell migration and scratch closure.

FIG. 4A-B. S. pyogenes growth curve in the presence of various concentrations of PhaNP@Syn71. The average of four replicates for each sample was computed together with standard deviations. (B) SEM images of S. pyogenes incubated with PhaNP@Syn71 compared to controls.

FIG. 5A-C. In vivo mouse wound model experiments with a combinatory treatment of intravenous (IV) injection followed by a daily topical dose of PhaNP@Syn71. (A) Normalized wound healing in mice treated with PhaNP@Syn71 vs controls. (B) Number of mice with wounds throughout the treatment. (C) Images of mice wounds at the start of treatment (Day 2), mid-treatment (Day 10) and the end of treatment with pluronic control (IV +topical), peptide control (IV+topical), peptide IV+PhaNP@Syn71 topical and PhaNP@Syn71 (IV+topical).

FIG. 6A-D. Histology samples of mice organs and skin tissue. (A)-(B) H&E staining on mouse organs and skin tissue was analyzed and scored for inflammation by a board-certified pathologist. The results showed no inflammation in any of the mice. Abbreviations: VV-Vehicle (IV+topical), PP-Peptide (IV+Topical), NP-Peptide (IV)+PhaNP@Syn71 (Topical), NN-PhaNP@Syn71 (IV+Topical). (C) Gram staining of the organs and skin tissue (D) SEM images of the organs and Skin tissue.

FIG. 7. DLS size distribution analysis on PhaNP and PhaNP@Syn71.FIG. 8. The extracted-ion chromatograms of (A) supernatant 1, (B) supernatant 2, and (C) standard of Syn71.

FIG. 9. Scratch closure assay scratch diameter change in time. While the Syn71 peptide promoted scratch closure, all the nanoparticle variants prevented any kind of cell migration due to their negative charge.

FIG. 10A-C. In vivo mouse wound model experiments with a treatment of daily topical dose only. (A) Normalized wound healing in mice treated with PhaNP@Syn71 vs controls (pluronic vehicle and Syn71 peptide). (B) Number of mice with wounds throughout the treatment. (C) Images of mice wounds at the end of treatment (Day 17) with vehicle control (V), peptide control (P) and PhaNP@Syn71 (N).

FIG. 11A-B. 20 nm nanoparticle characterization. (A) left) TEM image of SiNPs prior modifications with Au and Ag showing the uniform round shape and right) size distribution of SiNPs displaying an average diameter of 21.57 nm±1.63 nm. (B) left) TEM image of PhaNPs later modified with peptides confirming Au-Ag nanospheres present on the SiO₂ core nanoparticles and right) size distribution of PhaNPs showing an average diameter of 21.76 nm ±1.60 nm.

FIG. 12A-B. 95 nm nanoparticle characterization. (A) left) TEM image of SiNPs prior modifications with Au and Ag showing the uniform round shape and right) size distribution of SiNPs displaying an average diameter of 95.79 nm±7.43 nm. (B) left) TEM image of PhaNPs later modified with peptides confirming Au-Ag nanospheres present on the SiO₂ core nanoparticles and right) size distribution of PhaNPs showing an average diameter of 96.01 nm ±7.76 nm.

FIG. 13A-D. Growth curves of (A) P. aeruginosa FRD1, (B) S. aureus USA300, (C) A. baumannii and (D) K. pneumoniae in the presence of PhaNP@Syn20 with two different diameters (20 nm and 95 nm). Various concentrations (μM) of PhaNP@Syn20 were tested. For (A) top graphical line from left to right=P. aeruginosa FRD1 bacterial control; large diamond=8 μM Syn20 peptide; star=nanoparticle-Syn20 peptide [12.36 μM]; square=nanoparticle-Syn20 peptide [14.83 μM]; small diamond=nanoparticle-Syn20 peptide [18.54 μM]; hexagon=nanoparticle-Syn20 peptide [37.08 μM]; triangle=nanoparticle-Syn20 peptide [42.36 μM]; for (B) top graphical line from left to right=S. aureus USA300 bacterial control; large diamond=8 μM Syn20 peptide; small diamond=nanoparticle-Syn20 peptide [8 μM]; triangle=nanoparticle-Syn20 peptide [25 μM]; star=nanoparticle-Syn20 peptide [50 μM]; for (C) top graphical line from left to right=A. baumannii BAA-1605 bacterial control; large diamond=8 μM Syn20 peptide; small diamond=nanoparticle-Syn20 peptide [8 μM]; triangle=nanoparticle-Syn20 peptide [25 μM]; star=nanoparticle-Syn20 peptide [50 μM]; for (D) top graphical line from left to right=K. pneumoniae bacterial control; large diamond=8 μM Syn20 peptide; star=nanoparticle-Syn20 peptide [4 μM]; square=nanoparticle-Syn20 peptide [8 μM]; small diamond=nanoparticle-Syn20 peptide [10 μM]; hexagon=nanoparticle-Syn20 peptide [25 μM]; triangle=nanoparticle-Syn20 peptide [50 μM].

FIG. 14A-C. Growth curves of (A) P. aeruginosa FRD1 and (B) S. aureus USA300 in the presence of PhaNP@MC1-2 with diameter of 20 nm, and S. pyogenes in the presence of PhaNP@Syn71 with diameters of 20 nm and 95 nm. Various concentrations (μM) of PhaNP@Syn20 were tested. For (A) top graphical line from left to right=P. aeruginosa bacterial control; large diamond=8 μM MC1-2 peptide; star=nanoparticle-MC1-2 peptide [17.19 μM]; small diamond=nanoparticle-MC1-2 peptide [20.625 μM]; hexagon=nanoparticle-MC1-2 peptide [25.78 μM]; triangle=nanoparticle-MC1-2 peptide [51.56 μM]; for (B) top graphical line from left to right=S. aureus USA300 bacterial control; large diamond=8 μM MC1-2 peptide; star=nanoparticle-MC1-2 peptide [10.31 μM]; square=nanoparticle-MC1-2 peptide [17.19 μM]; small diamond=nanoparticle-MC1-2 peptide [20.625 μM]; hexagon=nanoparticle-MC1-2 peptide [25.78 μM]; triangle=nanoparticle-MC1-2 peptide [51.56 μM]; for (C) top graphical line from left to right=S. pyogenes bacterial control; large diamond=8 μM Syn71 peptide; star=nanoparticle-Syn71 peptide [4 μM]; square=nanoparticle-Syn71 peptide [8 μM]; small diamond=nanoparticle-Syn71 peptide [10 μM]; hexagon=nanoparticle-Syn71 peptide [25 μM]; triangle=nanoparticle-Syn71 peptide [50 μM].

FIG. 15. A single daily topical dose of PhANPs-Peptide 24 h post bacterial infection. S. aureus USA300 change in CFU/cm² of wound.

FIG. 16. A single daily topical dose of PhANPs-Peptide 24 h post bacterial infection. A. baumannii BA1605 change in CFU/cm² of wound.

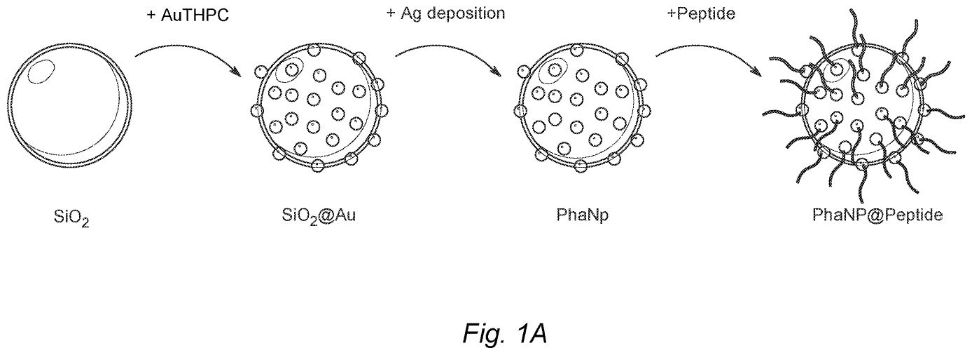

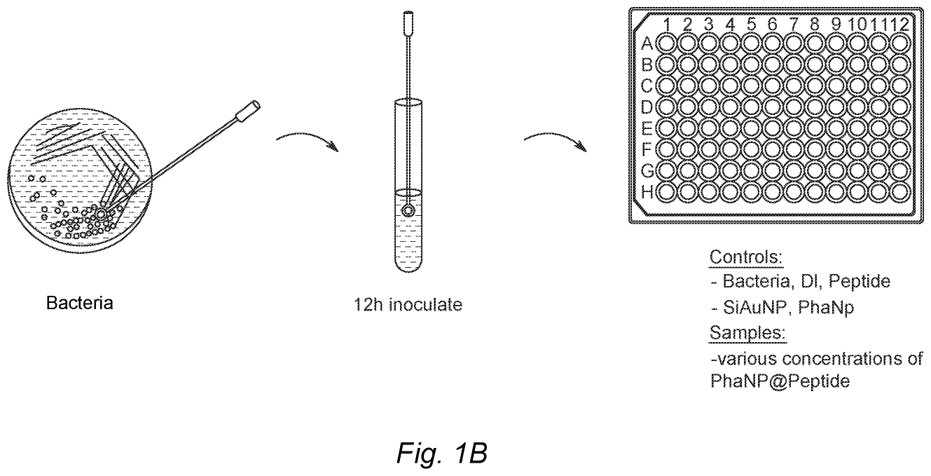

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, PCR amplification, cell culturing, etc.) are known in the art and described, for example, in Molecular Cloning: A Laboratory Manual, J. Sambrook et al., 4th edition, Cold Spring Harbor Laboratory Press, 2012.

In some instances, dosage information may be described, for example, in relation to certain non-human animals, such as mice or rabbits. Dosage conversion between non-human animals and humans are known in the art and described, for example, in Nair et al, J Basic Clin Pharma 2016; 7:27-31.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units is also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48:443-453) to find the global (i.e., spanning the complete sequences) alignment of two sequences that maximizes the number of matches and minimizes the number of gaps. The BLAST algorithm (Altschul et al. (1990) J Mol Biol 215:403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimize alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith TF, Waterman MS (1981) J. Mol. Biol 147 (1); 195-7).

Typically, this involves a first BLAST involving BLASTing a query sequence against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTS are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance).

Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbor joining tree, to help visualize clustering of related genes and to identify orthologues and paralogues.

The term “sequence identity” between two amino acid sequences is understood as meaning the percent identity of the amino acids sequence over in each case the entire sequence length which is calculated by alignment with the aid of the program algorithm GAP (Wisconsin Package Version 10.0, University of Wisconsin, Genetics Computer Group (GCG), Madison, USA), setting, for example, the following parameters: Gap Weight: 8; Length Weight: 2; Average Match: 2,912; Average Mismatch:-2,003.

### Embodiments of the Invention

Embodiments of the disclosure described herein antibacterial nanoparticles having biocidal and/or biostatic properties that can be useful in treating, for example, various infectious disease caused by bacterial infection, and in particular, antibiotic resistant bacterial strains. In some embodiments, the disclosure generally provides for antibacterial nanoparticles comprising: a silica inner core; one or more metal nanospheres surrounding the inner silica core; a surface layer comprising one or more metal coatings disposed on the metal nanospheres; and an antimicrobial peptide conjugated to the surface layer. Generally, a thiol or silane containing moiety may be introduced to one end of the antibacterial peptide to facilitate adsorption/conjugation to the surface layer (e.g., the silver surface layer or a surface layer comprising silver alloyed to a gold inner shell/layer) of the nanoparticle. Preferably, the thiol containing moiety is a cysteine amino acid incorporated at one end of the antibacterial peptide.

In some embodiments, the silica core comprises silicon dioxide SiO₂. The silica core may be synthesized, for example, the Stöber process involving hydrolysis and condensation of silica precursor tetraethyl orthosilicate (Si(OEt)₄, TEOS). The diameter of the resulting silica core 100 may be varied by varying the amount of ethanol, water, NH₄OH solution, TEOS, and the material of the reaction vessel (see Stober et al., J. Colloid Interface Sci. 26, 62-69 (1968)).

In some embodiments, the metal nanosphere surrounding the silica core comprises one or more of potassium, sodium, barium, calcium, manganese, chromium, cadmium, iron, nickel, tin, lead, antimony, bismuth, arsenic, mercury, silver, gold, copper, zinc, platinum, palladium, magnesium, aluminum, and combinations thereof. In some embodiments, the surface layer metal comprises one or more of potassium, sodium, barium, calcium, manganese, chromium, cadmium, iron, nickel, tin, lead, antimony, bismuth, arsenic, mercury, silver, gold, copper, zinc, platinum, palladium, magnesium, aluminum, and combinations thereof.

Exemplary silica, silver, and gold nanoparticles may be fabricated as described in U.S. Patent Publication No. 2021/0252162 to Nallathamby et al., incorporated herein by reference in its entirety. In some embodiments, the disclosure provides for a nanoparticle comprising: an inner silica core; a gold (Au) nanosphere surrounding the inner silica core; a surface layer comprising silver (Ag) surrounding the gold nanosphere or the surface layer comprises silver alloyed into the gold nanosphere; and an antimicrobial peptide conjugated to the surface layer, wherein the antimicrobial peptide is selected from the group consisting of a cysteine terminated Syn71 peptide or derivative thereof, a cysteine terminated MC1-2 peptide, and a cysteine terminated Syn20 peptide.

In other embodiments, an antibacterial nanoparticle comprises an inner silica core; a gold (Au) nanosphere surrounding the inner silica core; a surface layer comprising silver (Ag) surrounding the gold nanosphere; and an antimicrobial peptide conjugated to the surface layer, wherein the antimicrobial peptide is selected from the group consisting of a cysteine terminated Syn71 peptide, a cysteine terminated MC1-2 peptide, and a cysteine terminated Syn20 peptide.

In some embodiments, an antibacterial nanoparticle comprises a silica core; a plurality of silver-coated gold nanospheres conjugated to the silica core; and an antimicrobial peptide conjugated to the plurality of silver-coated gold nanospheres, wherein the antimicrobial peptide is selected from the group consisting of a cysteine terminated Syn71 peptide, a cysteine terminated MC1-2 peptide, and a cysteine terminated Syn20 peptide.

In some embodiments, an antibacterial nanoparticle comprises an inner silica core and an alloyed surface layer comprising an inner gold (Au) layer and an outer silver (Ag) layer. In other embodiments, an antibacterial nanoparticle comprises an inner silica core and an alloyed surface layer comprising an alloy of gold (Au) layer and silver (Ag), wherein the outer most surface of the surface layer substantially comprises silver. The antibacterial peptide is also conjugated to the outer most surface of the surface layer.

In other embodiments, an antibacterial nanoparticle comprises an inner silica core and a plurality of metal shells forming a surface layer, wherein the plurality of metal shells surrounding the inner silica core comprise an alloy of gold (Au) and silver (Ag). The antibacterial peptide is also conjugated to the outer most surface of the surface layer.

In some embodiments, the cysteine terminated Syn71 peptide or derivative thereof comprises an amino acid sequence of CAGTEKIFQRLKKTIQEGKKIAKRWW (SEQ ID NO: 1).

In other embodiments, the cysteine terminated Syn71 peptide or derivative thereof is described in described in U.S. Patent Publication No. 2021/0252162 to Nallathamby et al., incorporated herein by reference in its entirety. Other antibacterial peptides that may be used with embodiments of the invention are described, for example, in U.S. Pat. No. 10,676,721 to Collins et al.

In some embodiments, the antibacterial peptide is the cysteine terminated MC1-2 peptide, wherein the cysteine terminated MC1-2 peptide comprises an amino acid sequence of CSAVWRHWRRFWLRKRK (SEQ ID NO: 2).

In some embodiments, the antibacterial peptide is the cysteine terminated Syn20 peptide, wherein the cysteine terminated Syn20 peptide comprises an amino acid sequence of CAWKKTIRQYLKNKIKKKGRKAVIAW (SEQ ID NO: 3).

In another embodiments, an antibacterial peptide may include one or more of the following: Syn-safencin_92_Cys CAWKKTIRQYLKNKIKKKWRKAVIAW (SEQ ID NO: 11); Syn-safencin_96_Cys CAWKEKIRKKLKNEIKKKWRKAVIAW (SEQ ID NO: 12); AIP_1_Cys FTMKKSLLLLFFLGTINFSLC (SEQ ID NO: 13); Chenisirin_cys CSAVWRWRRFWLRKRK (SEQ ID NO: 14); E16LKL_cys CKGLKKLLKLLKKLLKL (SEQ ID NO: 15); MC1_2-cys CSAVWRWRRFWLRKRK (SEQ ID NO: 16); and E14KK1_cys CLKKLLKKLKKLLKK (SEQ ID NO: 17). In some embodiments, the nanoparticles have an average diameter of about 5 nm to about 150 nm, about 10 nm to about 125 nm, about 15 nm to about 100 nm, about 20 nm to about 90 nm, about 25 nm to about 80 nm, about 30 nm to about 70 nm, about 35 nm to about 60 nm, or about 40 nm to about 50 nm. In other embodiments, the nanoparticles have an average diameter of about 5 nm, about 10 nm, about 15 nm, about 20 nm, about 25 nm, about 30 nm, about 35 nm, about 40 nm, about 50 nm, about 55 nm, about 60 nm, about 65 nm, about 70 nm, about 75 nm, about 80 nm, about 85 nm, about 90 nm, about 95 nm, about 100 nm, about 105 nm, or about 110 nm. In some embodiments, the nanoparticles have an average diameter of about 15 nm to about 20 nm or about 90 nm to about 100 nm. In other embodiments, the nanoparticles have an average diameter of about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, or about 30 nm. In other embodiments, the nanoparticles have an average diameter of to about 90 nm, about 91 nm, about 92 nm, about 93 nm, about 94 nm, about 95 nm, about 96 nm, about 97 nm, about 98 nm, about 99 nm, or about 100 nm. In some embodiments, an antibacterial peptide (bactericidal or bacteriostatic) is conjugated to the surface layer of the nanoparticle. Preferably, the surface layer comprises silver (Ag) or a plurality of silver shells surrounding the inner core or shells of the nanoparticle. In some embodiments, the antibacterial peptide is conjugated to the surface layer in an average concentration of about 0.1 μM to about 200 μM, about 0.25 μM to about 200 μM, about 1 μM to about 200 μM, about 1 μM to about 175 μM, about 1 μM to about 150 μM, about 5 μM to about 150 μM, about 10 μM to about 150 μM, about 15 μM to about 150 μM, about 20 μM to about 150 μM, about 25 μM to about 150 μM, about 30 M to about 150 μM, about 35 μM to about 150 μM, about 40 μM to about 150 μM, about 45 μM to about 150 μM, about 50 μM to about 150 μM, about 55 μM to about 150 μM, about 60 μM to about 150 μM, about 65 μM to about 150 μM, about 70 μM to about 150 μM, about 75 μM to about 150 μM, about 80 μM to about 150 μM, about 85 μM to about 150 μM, about 90 μM to about 150 μM. about 95 μM to about 150 μM, about 100 μM to about 150 μM, about 105 μM to about 150 μM, about 110 μM to about 150 μM, about 115 μM to about 150 μM, about 120 μM to about 150 μM, about 125 μM to about 150 μM, about 130 μM to about 150 μM, about 135 μM to about 150 μM, about 140 μM to about 150 μM, or about 145 μM to about 150 μM. In other embodiments, the antibacterial peptide is conjugated to the surface layer in an average concentration of about 40 μM to about 100 μM. In other embodiments, the antibacterial peptide is conjugated to the surface layer in an average concentration of about 40 μM to about 60 μM or about 80 μM to about 100 μM.

In some embodiments, a Syn71 peptide is conjugated to the surface layer of the nanoparticle at an average concentration of about 0.1 μM to about 175 μM, about 0.25 μM to about 175 μM, about 1 μM to about 175 μM, about 1 μM to about 150 μM, about 1 μM to about 125 M, or about 1 μM to about 100 μM. In other embodiments, a Syn71 peptide is conjugated to the surface layer of the nanoparticle at an average concentration of about 50 μM to about 175 μM, about 75 μM to about 150 μM, or about 100 μM to about 150 μM. In other embodiments, a Syn71 peptide is conjugated to the surface layer of the nanoparticle at an average concentration of about 40 μM to about 60 μM, or about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, or about 60 μM.

In some embodiments, a MC1-2 peptide is conjugated to the surface layer of the nanoparticle at an average concentration of about 0.1 μM to about 175 μM, about 0.25 μM to about 175 μM, about 1 μM to about 175 μM, about 1 μM to about 150 μM, about 1 μM to about 125 μM, or about 1 μM to about 100 μM. In other embodiments, a Syn71 peptide is conjugated to the surface layer of the nanoparticle at an average concentration of about 50 μM to about 175 μM, about 75 μM to about 150 μM, or about 100 μM to about 150 μM. In some embodiments, an MC1-2 peptide is conjugated to the surface layer of the nanoparticle at an average concentration of about 40 μM to about 60 μM, or about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, or about 60 μM.

In some embodiments, a Syn20 peptide is conjugated to the surface layer of the nanoparticle at an average concentration of about 0.1 μM to about 175 μM, about 0.25 μM to about 175 μM, about 1 μM to about 175 μM, about 1 μM to about 150 μM, about 1 μM to about 125 μM, or about 1 μM to about 100 μM. In other embodiments, a Syn20 peptide is conjugated to the surface layer of the nanoparticle at an average concentration of about 50 μM to about 175 μM, about 75 μM to about 150 μM, or about 100 μM to about 150 μM. In some embodiments, a Syn20 peptide is conjugated to the surface layer of the nanoparticle at an average concentration of about 40 μM to about 60 μM, or about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, or about 60 μM.

The disclosure also provides for compositions comprising a population of antibacterial nanoparticle and pharmaceutically acceptable excipient or carrier, wherein the antibacterial nanoparticles comprise: an inner silica core; a gold (Au) nanosphere surrounding the inner silica core; a surface layer comprising silver (Ag) surrounding the gold nanosphere or the silver is alloyed into the gold nanosphere; and an antimicrobial peptide conjugated to the surface layer, wherein the antimicrobial peptide is selected from the group consisting of a cysteine terminated Syn71 peptide or derivative thereof, a cysteine terminated MC1-2 peptide, and a cysteine terminated Syn20 peptide.

In some embodiments, the composition comprises a population of antibacterial nanoparticles having a Syn71 peptide, a Syn20 peptide, or a MC1-2 peptide conjugated to the surface layer at an average concentration of about 0.25 μM to about 150 μM, about 1 μM to about 150 μM, about 5 μM to about 145 μM, about 10 μM to about 140 μM, about 15 μM to about 135 μM, about 20 μM to about 130 μM, about 25 μM to about 125 μM, about 30 μM to about 120 M, about 35 μM to about 115 μM, about 40 μM to about 110 μM, about 45 μM to about 105 M, about 50 μM to about 100 μM, about 55 μM to about 95 μM, about 60 μM to about 90 μM, about 65 μM to about 85 μM, or about 70 μM to about 80 μM. In some embodiments, the antibacterial peptide is conjugated to the surface layer at an average concentration of about 1 μM to about 150 μM or 75 μM to about 150 μM, In another embodiment, the population of antibacterial nanoparticles have a Syn71 peptide, a Syn20 peptide, or a MC1-2 peptide conjugated to the surface layer at an average concentration of about 1 μM to about 75 μM.

In some embodiments the population of antibacterial nanoparticles comprises the Syn71 peptide, the Syn20 peptide, or the MC1-2 peptide conjugated to the surface layer of the nanoparticle at an average concentration of about 40 μM to about 60 μM, or about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, or about 60 μM.

In some embodiments, a nanoparticle comprises an inner silica core; a gold (Au) nanosphere surrounding the inner silica core; a surface layer comprising silver (Ag) surrounding the gold nanosphere or the surface layer comprises silver alloyed into the gold nanosphere; and an antimicrobial peptide conjugated to the surface layer, wherein the antimicrobial peptide is selected from any one of SEQ ID NO: 1-3 and SEQ ID NO: 11-17.

In some preferred embodiments, the nanoparticles have an average diameter of about 15 nm to about 30 nm or about 90 nm to about 100 nm. In other embodiments, the nanoparticles have an average diameter of about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, or about 30 nm. In other embodiments, the nanoparticles have an average diameter of to about 90 nm, about 91 nm, about 92 nm, about 93 nm, about 94 nm, about 95 nm, about 96 nm, about 97 nm, about 98 nm, about 99 nm, or about 100 nm.

The disclosure also provides for methods of treating a bacterial infection comprising contacting a bacterial cell with an effective amount of the antibacterial nanoparticle or the composition described herein, wherein the antibacterial nanoparticle reduces growth of the bacterial cell by greater than about 95% and/or kills the bacterial cell.

In some embodiments, the antibacterial nanoparticles are bacteriostatic and reduce growth of the bacterial cell by greater than about 90%, about 91%, about 92%, about 93%, about 94%, 95%, about 96%, about 97%, about 98%, about 99%, or about 99.99%. In some embodiments, the antibacterial nanoparticles are bactericidal and kills greater than about 80%, about 85%, about 90%, about 91%, about 92%, about 93%, about 94%, 95%, about 96%, about 97%, about 98%, about 99%, or about 99.99% of the bacterial cells.

In some embodiments, the bacterium is selected from among Acinetobacter spp.; Actinobaccillus spp.; Actinomadura spp.; Actinomyces spp.; Actinoplanes spp.; Aeromonas spp.; Agrobacterium spp.; Alistipes spp.; Anaerococcus spp.; Arthrobacter spp.; Bacillus spp.; Brucella spp.; Bulleidia spp.; Burkholderia spp.; Cardiobacterium spp.; Citrobacter spp.; Clostridium spp.; Corynebacterium spp.; Dermatophilus spp.; Dorea spp.; Edwardsiella spp.; Enterobacter spp.; Enterococcus spp.; Erysipelothrix spp.; Escherichia spp.; Eubacterium spp.; Faecalibacterium spp.; Filifactor spp.; Finegoldia spp.; Flavobacterium spp.; Gallicola spp.; Haemophilus spp.; Helcococcus spp.; Holdemania spp.; Hyphomicrobium spp.; Klebsiella spp.; Lactobacillus spp.; Legionella spp.; Listeria spp.; Methylobacterium spp.; Micrococcus spp.; Micromonospora spp.; Mobiluncus spp.; Moraxella spp.; Morganella spp.; Mycobacterium spp.; Neisseria spp.; Nocardia spp.; Paenibacillus spp.; Parabacteroides spp.; Pasteurella spp.; Peptoniphilus spp.; Peptostreptococcus spp.; Planococcus spp.; Planomicrobium spp.; Plesiomonas spp.; Porphyromonas spp.; Prevotella spp.; Propionibacterium spp.; Proteus spp.; Providentia spp.; Pseudomonas spp.; Ralstonia spp.; Rhodococcus spp.; Roseburia spp.; Ruminococcus spp.; Salmonella spp.; Sedimentibacter spp.; Serratia spp.; Shigella spp.; Solobacterium spp.; Sphingomonas spp.; Sporanaerobacter spp.; Staphylococcus spp.; Stenotrophomonas spp.; Streptococcus spp.; Streptomyces spp.; Tissierella spp.; Vibrio spp.; and Yersinia spp.

In some embodiments, the bacterial cell causing the bacterial infection comprises one or more of Enterococcus faecalis, Corynebacterium striatum, Staphylococcus aureus, Klebsiella pneumoniae, Acinetobacter baumannii, Pseudomonas aeruginosa, and Streptococcus pyogenes. In some embodiments, the bacterial cell is one or more of Enterococcus faecalis, Enterococcus faecium, Enterococcus avium, Enterococcus gallinarum, Enterococcus casseliflavus, Enterococcus durans, Enterococcus raffinosus, and Enterococcus mundtii. In some embodiments, the bacterial cell is Enterococcus faecalis. In some embodiments, the bacterial cell is one or more of Corynebacterium diphtheriae, Corynebacterium jeikeium, Corynebacterium striatum, and Corynebacterium urealyticum. In some embodiments, the bacterial cell is Corynebacterium striatum. In some embodiments, the bacterial cell is one or more of Staphylococcus argenteus, Staphylococcus aureus, Staphylococcus schweitzeri, and Staphylococcus simiae. In some embodiments, the bacterial cell is Staphylococcus aureus. In some embodiments, the bacterial cell is one or more of Klebsiella aerogenes, Klebsiella granulomatis, Klebsiella grimontii, Klebsiella huaxiensis, Klebsiella kielensis, Klebsiella michiganensis, Klebsiella milletis, Klebsiella oxytoca, Klebsiella pneumoniae, Klebsiella quasipneumoniae subsp. Quasipneumoniae or subsp. Similipneumoniae, Klebsiella quasivariicola, Klebsiella senegalensis, Klebsiella steroids, and Klebsiella variicola. In some embodiments, the bacterial cell is Klebsiella pneumoniae. In some embodiments, the bacterial cell is one or more of Acinetobacter albensis, Acinetobacter apis, Acinetobacter baumannii, Acinetobacter baylyi, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter celticus, Acinetobacter chengduensis, Acinetobacter colistiniresistens, Acinetobacter courvalinii, Acinetobacter cumulans, Acinetobacter defluvii, Acinetobacter dispersus, Acinetobacter dijkshoorniae, Acinetobacter equi, Acinetobacter gandensis, Acinetobacter gerneri, Acinetobacter guangdongensis, Acinetobacter guerrae, Acinetobacter guillouiae, Acinetobacter gyllenbergii, Acinetobacter haemolyticus, Acinetobacter harbinensis, Acinetobacter indicus, Acinetobacter junii, Acinetobacter kookii, Acinetobacter lactucae, Acinetobacter lanii, Acinetobacter larvae, Acinetobacter lwoffii, Acinetobacter modestus, Acinetobacter nectaris, Acinetobacter nosocomialis, Acinetobacter oryzae, Acinetobacter parvus, Acinetobacter pakistanensis, Acinetobacter populi, Acinetobacter portensis, Acinetobacter proteolyticus, Acinetobacter pittii, Acinetobacter piscicola, Acinetobacter pragensis, Acinetobacter proteolyticus, Acinetobacter pseudolwoffii, Acinetobacter pullicarnis, Acinetobacter pullorum, Acinetobacter puyangensis, Acinetobacter qingfengensis, Acinetobacter radioresistens, Acinetobacter rudis, Acinetobacter schindleri, Acinetobacter seifertii. Acinetobacter shaoyimingii, Acinetobacter soli, Acinetobacter stercoris, Acinetobacter tandoii, Acinetobacter tjernbergiae, Acinetobacter towneri, Acinetobacter ursingii, Acinetobacter variabilis, Acinetobacter venetianus, Acinetobacter vivianii, Acinetobacter wanghuae, and Acinetobacter wuhouensis. In some embodiments, the bacterial cell is Acinetobacter baumanni. In some embodiments, the bacterial cell is one or more of Pseudomonas alcaligenes, Pseudomonas mendocina, Pseudomonas pseudoalcaligenes, Pseudomonas resinovorans, Pseudomonas aeruginosa, Pseudomonas citronellolis, Pseudomonas citronellolis, Pseudomonas delhiensis, Pseudomonas denitrificans, Pseudomonas jinjuensis, Pseudomonas knackmussii, Pseudomonas nicosulfuronedens, Pseudomonas nitroreducens, Pseudomonas panipatensis Pseudomonas putida, Pseudomonas desmolyticum, and Pseudomonas Nitroreducens, and Pseudomonas veronii. In some embodiments, the bacterial cell is Pseudomonas aeruginosa. In some embodiments, the bacterial cell is one or more of Streptococcus agalactiae, Streptococcus bovis, Streptococcus mutans, Streptococcus pneumoniae, Streptococcus pyogenes, Streptococcus salivarius, Streptococcus sanguinis, Streptococcus suis, Streptococcus viridans, and Streptococcus uberis; In some embodiments, the Streptococcus is Streptococcus pyogenes.

The disclosure also provides for methods of treating a bacterial infection in a subject comprising administering an effective amount of the antibacterial nanoparticle or the composition as described herein to the subject in need thereof, wherein the bacterial infection is of a wound or implant material.

The disclosure also provides for medical products that incorporate the antibacterial nanoparticles or a composition comprising said antibacterial nanoparticles. In some embodiments, the medical product comprises a bandage or wound dressing. In some embodiments, the bandage or wound dressing is fabricated with the antibacterial nanoparticles embedded or impregnated within the structure of the bandage or wound dressing (e.g., between or on several layers of absorbent material than makes up the bandage or wound dressing). In some embodiments, the bandage or wound dressing may comprise a population of the antibacterial nanoparticles having an average concentration the antibacterial peptide conjugated to the surface layer of about 1 μM to about 200 μM, or about 50 μM to about 150 μM, or about 75 μM to about 150 μM. In other embodiments, the population of the antibacterial nanoparticles have an average concentration of the antibacterial peptide conjugated to the surface layer of about 50 μM to about 60 μM.

In other embodiments, the antibacterial nanoparticles are provided as a medicament (e.g., an ointment or cream) that can be applied to the surface of the bandage or wound dressing prior to contacting the wound. In some embodiments, the medicament may comprise a population of the antibacterial nanoparticles having an average concentration the antibacterial peptide conjugated to the surface layer of about 1 μM to about 200 μM, or about 50 μM to about 150 μM, or about 75 μM to about 150 μM. In other embodiments, the population of the antibacterial nanoparticles in the medicament have an average concentration of the antibacterial peptide conjugated to the surface layer of about 50 μM to about 60 μM.

In some embodiments, the medical product is one or more of a medical implant, a dental implant, and a surgical instrument. In some embodiments, the implants may be a prostheses, an implant as artificial substitutes for body parts, or materials inserted into tissue for functional, cosmetic, or therapeutic purposes. Prostheses can be functional, as in the case of artificial arms and legs; cosmetic, as in the case dermal filler; and therapeutic as in the case of implants surgically inserted or grafted into the body and intended to replace non-functioning organs. Exemplary medical implants further include vascular devices such as grafts (e.g., abdominal aortic aneurysm grafts, etc.), stents, catheters (including arterial, intravenous, blood pressure, stent graft, etc.), valves (e.g., polymeric or carbon mechanical valves,), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies, peripheral cardiovascular devices, atrial septal defect closures, left atrial appendage filters, valve annuloplasty devices, mitral valve repair devices, vascular intervention devices, ventricular assist pumps, and vascular access devices (including parenteral feeding catheters, vascular access ports, central venous access catheters); surgical devices such as sutures of all types, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices such as scleral buckles and sponges, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuro-aneurysm treatment coils, and neuropatches, splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; oncological implants; and pain management implants. Exemplary dental implants include root devices, such as those made from titanium, used to support restorations that resemble a tooth or group of teeth to replace missing teeth.

Other examples of non-organic surfaces that may be coated or impregnated contemplated by the present invention include surfaces of medical supplies and medical equipment. Non-limiting examples of medical supplies and equipment include gloves (such as disposable gloves), gowns, pads, wheelchairs, stretchers, tables, swabs, sponges, sutures (such as silk sutures), bags, surgical supplies, and packaging materials for the packaging of sterile medical or hospital supplies. Further examples of non-organic surfaces include the surfaces of a sponge, wipe, pad, or mop.

Exemplary surgical instruments include Scalpels; Forceps: gullet forceps, bone clamping forceps, right angle forceps, abortion forceps, intestinal forceps, hemostatic forceps, artery forceps, towel forceps for scalp, ablation forceps, thread forceps, bulldog forceps for implantation, Fogarty blood vessel forceps, hemoclip forceps, gastric forceps, Lister forceps, caval forceps, duodenal forceps, bone forceps, stump appendix forceps, arthrosis forceps, tendon inducing forceps, thyroid forceps, lymphatic gland forceps, pleurolysis forceps, bone rongeur forceps, babcock forceps, multipurpose blood vessel forceps, curette forceps, connective tissue compressive forceps, mosquito forceps, Küstner uterine safety forceps, blood vessel forceps, Pean forceps, Martin tenaculum forceps, drain forceps, Kocher's forceps, Muzeaux tenaculum forceps, Melabulldog forceps, Kelly's forceps, forceps with shank, thoraco- or laparo-scopic forceps, Allis forceps, Bulldog forceps, Mikulicz forceps, towel forceps, placenta forceps, etc.; Scalpel holders; Elevatoriums; Raspatriums; Hammers; Rongeurz; Spatulas: enteric spatula, cerebral spatula, nervous spatula, etc.; Luer; Tweezers; Retractors: Adson, Gelpi, Weitlaner, etc.; Mouth gags; Retractors: muscle retractor, Tensho type sharp retractor, nerve retractor, lung hemorrhage retractor, saddle retractor, extra-large width vastus muscle retractor, Diva retractor, Hohmann retractor, scapula retractor, ureteral retractor, tendon traction retractor, maxillary sinuses retractor, flat retractor, demar retractor, etc.; Kerrisons; Scissors: ophthalmic scissors, plastic surgery scissors, scanlan scissors, backward rib scissors, etc.; Drills; Needle holders: Mathieu, Scamed, Sarot, Rider, Webster, Sweden, etc.; Spreaders; Hooks: skin hook, French hook, etc.; Clips: Lenin Clip, etc.; Towels: cup, etc.; Retractors; Metal Petri dishes; Threads; Masks (for doctor, nurse, for ventilation of patients; Gloves; Surgical gowns; Gauzes; Needles: injection needle, aneurysm needle, cerebral ventricles tap needle, Deschamps aneurysm needle, etc.; Sondes; Beakers; Trays; Pus basins; Curettes: soft curette, dental bone curette; Rasoriums: dental rasorium, nerve rasorium, oral cleft rasorium, otolaryngological rasorium, etc.; Endoscopes: anal speculum, skull endoscope, nasal speculum, vaginal speculum, aural speculum, arthroscope, rectal speculum, capsule endoscope, etc.; Forceps: blood vessel forceps, Sweden forceps, Bergh forceps, microforceps, otolaryngological forceps, cushing forceps, dental forceps, hook forceps, bent nose DeBakey forceps long forceps, Adson forceps, Prince forceps, etc.; Tongue depressors; Suction tubes; Files; Mirrors; Stethoscopes; Scissors: Cooper, Mecchen, etc.; and Scissors (also see, for example, U.S. Pat. No. 8,877,222 to Aizawa et al.).

In some embodiments, the antibacterial nanoparticle is disposed on a surface of the medical implant, the dental implant, and the surgical instrument. In some embodiments, the population of antibacterial nanoparticles disposed on the surface of the medical or dental implant or the surgical instruments may comprise an average concentration of the antibacterial peptide conjugated to the surface layer of about 1 μM to about 200 μM, or about 50 μM to about 150 μM, or about 75 μM to about 150 μM.

In some embodiments, a topical composition may comprise block copolymers of polyethylene glycol and polypropylene glycol including poloxamers. Poloxamers are nonionic triblock copolymers composed of a central hydrophobic chain of polyoxypropylene (poly(propylene oxide)) flanked by two hydrophilic chains of polyoxyethylene (poly(ethylene oxide)). Exemplary poloxamers include, but not limited to, poloxamer 124, poloxamer 188, poloxamer 237, poloxamer 338, poloxamer 407 and the like. In a preferred embodiment, the poloxamer is poloxamer 407 (i.e., Pluronic® F-127).

In some embodiments, a topical composition may comprise about 1% w/v to about 25% w/v poloxamer and a population of nanoparticles having a peptide concentration on the surface of the nanoparticle of about 0.1 μM to about 150 μM, about 5 μM to about 150 M, about 10 μM to about 150 μM, about 20 μM to about 150 μM, about 25 μM to about 150 μM, about 50 μM to about 150 μM, or about 75 μM to about 150 μM.

In some embodiments, a topical composition may comprise about 1% w/v to about 25% w/v poloxamer 407 and a population of nanoparticles having a peptide concentration on the surface of the nanoparticle of about 0.1 μM to about 150 μM, about 5 μM to about 150 μM, about 10 μM to about 150 μM, about 20 μM to about 150 μM, about 25 μM to about 150 μM, about 50 μM to about 150 μM, or about 75 μM to about 150 μM.

In some embodiments, the topical composition may comprise about 1% w/v to about 25% w/v poloxamer 407, about 5% w/v to about 25% w/v poloxamer 407, about 10% w/v to about 25% w/v poloxamer 407, about 15% w/v to about 25% w/v poloxamer 407, or about 20% w/v to about 25% w/v poloxamer 407. In some embodiments, the topical composition may comprise about 20% w/v poloxamer 407.

In some embodiments, a composition may comprise about 0.1% w/v silica, about 0.5% w/v silica, about 1% w/v silica, about 1.5% w/v silica, about 2% w/v silica, about 2.5% w/v silica, about 3% w/v silica, about 3.5% w/v silica, about 4% w/v silica, about 4.5% w/v silica, or about 5% w/v silica. In other embodiments, a composition may comprise about 0.1% w/v to about 10% w/v silica, or about 0.1% w/v to about 5% w/v silica.

In some embodiments, antibacterial nanoparticles are administered parenterally. Parenteral administration includes one or more of subcutaneous, intramuscular, intravenous, and intrathecal administration. In some embodiments, antibacterial nanoparticles may be administered intravenously. In other embodiments, the antibacterial nanoparticles may be added to a topological cream (e.g., an ointment or cosmetic cream) for treating bacterial infections by contacting the bacteria.

In some embodiments, the amount of antibacterial nanoparticles present in a composition may be about 0.001 mg to about 5000 mg, about 0.01 mg to about 1000 mg, about 0.1 mg to about 500 mg, or about 1 mg to about 100 mg. In some embodiments, a dose of nanoparticles may be administered parenterally in an mount of about 0.5 mg/kg to about 1000 mg/kg, about 0.5 mg/kg to about 950 mg/kg, about 0.5 mg/kg to about 900 mg/kg, about 0.5 mg/kg to about 850 mg/kg, about 0.5 mg/kg to about 800 mg/kg, about 0.5 mg/kg to about 750 mg/kg, about 0.5 mg/kg to about 700 mg/kg, about 0.5 mg/kg to about 650 mg/kg, about 0.5 mg/kg to about 600 mg/kg, about 0.5 mg/kg to about 550 mg/kg, about 0.5 mg/kg to about 500 mg/kg, about 0.5 mg/kg to about 450 mg/kg, about 0.5 mg/kg to about 400 mg/kg, about 0.5 mg/kg to about 350 mg/kg, about 0.5 mg/kg to about 300 mg/kg, about 0.5 mg/kg to about 250 mg/kg, about 0.5 mg/kg to about 100 mg/kg, about 0.5 mg/kg to about 75 mg/kg, about 0.5 mg/kg to about 50 mg/kg, or about 0.5 mg/kg to about 25 mg/kg. In some embodiments, a dosage is administered parenterally in amount of about 10 mg/kg to about 350 mg/kg.

### Pharmaceutical Formulations.

The nanoparticles described herein can be used to prepare therapeutic pharmaceutical compositions, for example, by combining the nanoparticles with a pharmaceutically acceptable diluent, excipient, or carrier.

The nanoparticles of the formulas described herein can be formulated as pharmaceutical compositions and administered to a mammalian host, such as a human patient, in a variety of forms. The forms can be specifically adapted to a chosen route of enteral administration, e.g., oral administration, sublingual administration, or rectal administration.

The nanoparticles described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the nanoparticles may be incorporated into sustained-release preparations and devices.

Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms include aqueous solutions, dispersions, gels (e.g., poloxamer 407 or Pluronic® F-127, or sterile powders comprising the active ingredient, optionally encapsulated in liposomes. The ultimate dosage form should be sterile, fluid and stable under the conditions of manufacture and storage. A liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the compositions can be brought about by agents capable of delaying absorption, for example, aluminum monostearate and/or gelatin.

Various dosage forms can be prepared by incorporating the nanoparticles in the required amount in the appropriate solvent with various other ingredients enumerated above, optionally followed by filter sterilization. Methods of preparation can include vacuum drying and freeze-drying techniques, which yield a powder of the active ingredient plus any additional desired ingredient present in the solution.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be administered orally or sprayed into the mouth using a pump-type or aerosol sprayer. Thickeners such as synthetic polymers, fatty acids, fatty acid salts and esters, fatty alcohols, modified celluloses, or modified mineral materials can also be employed with liquid carriers.

Useful dosages of the nanoparticles described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 500 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The nanoparticle is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The nanoparticles can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Example 1. Syn71 Nanoparticles

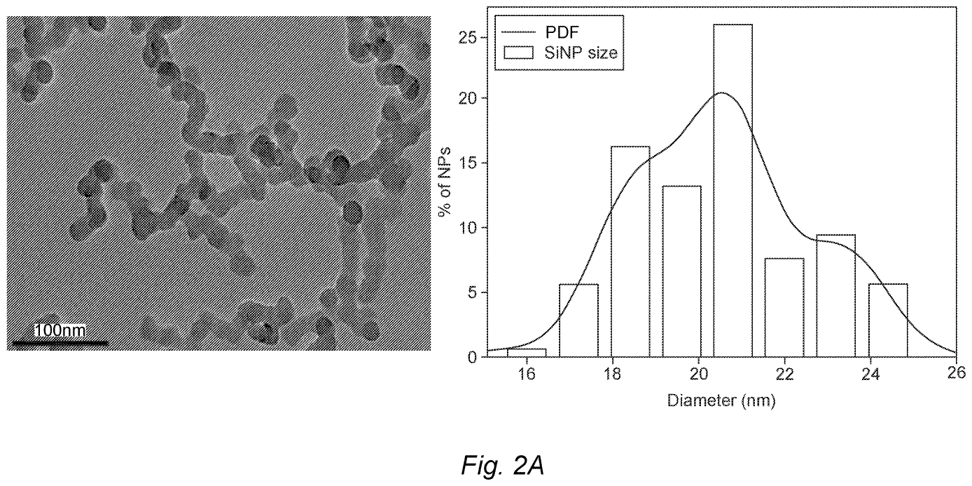

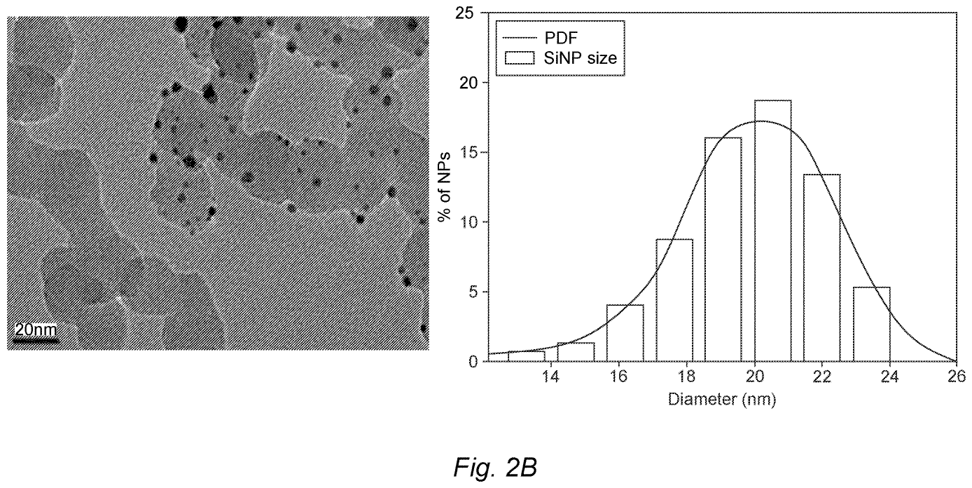

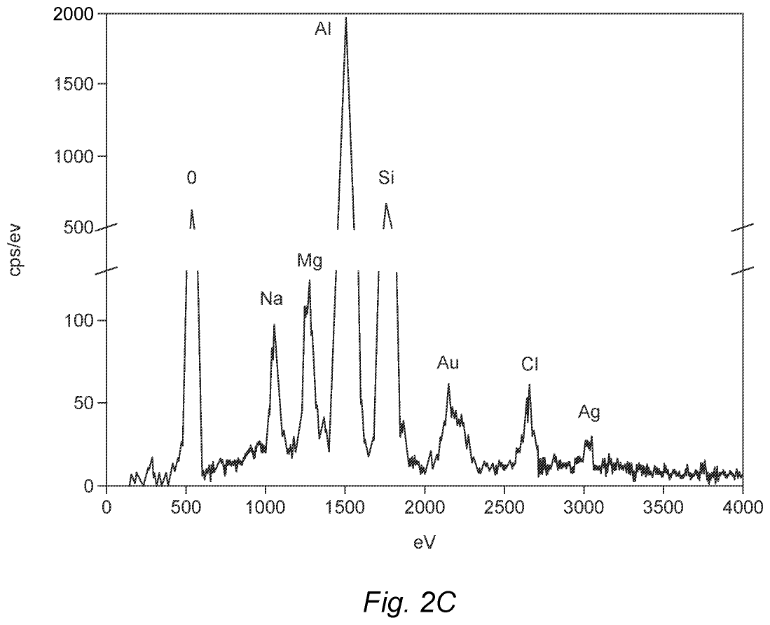

Nanoparticle characterization. Previous work done by our research group 37 has confirmed that the structure of our synthesized nanoparticles resembles naturally occurring bacteriophages, with smaller-sized nanoparticles dis-playing more close mimicry with bacteriophages. The nanoparticles mimic bacteriophages from the family of Microviridae with 88% similarity. Microviridae are tailless bacteriophages, sized 25-27 nm. To visualize the PhaNPs, TEM analysis was carried out (FIG. 2A-B), revealing that the diameter of SiO₂ nanoparticle core was 20.47 nm±2.14 nm (FIG. 2A). Analysis of PhaNP@Syn71 showed a nanoparticle diameter of 20.18 nm±2.41 nm (FIG. 2B), verifying that further synthesis processes (addition of gold nanospheres, silver and Syn71 peptide) do not alter the base diameter of the nanoparticle silica core. In addition, TEM image analysis of PhaNPs and PhaNP@Syn71 proved the presence of gold NPs on the silica core.

EDXS compositional analysis of the PhaNPs (FIG. 2C) revealed the presence of Si, Au and Ag, in agreement with the synthesis process, with ICP-OES measurements (Table A) further confirming the presence of Au on SiAuNPs and Au and Ag on PhaNPs. The concentration of Si was 3.829 mg/ml and 12.068 mg/ml for SiAuNPs and PhaNPs, respectively. The concentration of Au on SiAuNPs were 0.124 mg/ml while the Au and Ag concentrations of PhaNPs were 0.054 mg/ml and 3.924 mg/ml, respectively. This is consistent with the synthesis process, showing similar gold con-tent of SiAuNPs and PhaNPs, with the addition of Ag on PhaNPs. DLS analysis was carried out on PhaNPs without the Syn71 peptide and PhaNP@Syn71 peptide modified nanoparticles. The results showed an average diameter of the nanoparticles to be 781.7±498.5 nm, indicating that the PhaNPs aggregate in PBS solvent (Table B).

PhaNP@Syn71 had an average diameter of 2582±641.2 nm, showing an increase in aggregation compared to PhaNPs. These results indicate the actual be-haviour of the nanoparticles in the PBS solution. The zeta potential results were 16.0 mV for PhaNPs with a conductivity of 17.8 mS/cm, while the zeta potential for PhaNP@Syn71 was 8.54 mV with a conductivity of 16.8 mS/cm, indicating an increase in surface potential when the PhaNPs are modified with Syn71 peptide. Both zeta potential and DLS analyses confirmed the Syn71 peptide addition to the PhaNP surface, showing the change in zeta potential as well as the size, which has been shown also in previously published works to indicate successful peptide addition to the nanoparticles.

TABLE A
ICP-OES of nanoparticles at different synthesis steps to
quantitatively confirm the presence of the expected elements.

| | Si (mg/ml) | Au (mg/ml) | Ag (mg/ml) |
|---|---|---|---|
| SiNP | 7.371 | 0 | 0 |
| SiAuNP | 3.829 | 0.124 | 0 |
| PhaNP | 12.068 | 0.054 | 3.924 |

TABLE B
DLS zeta potential.

| | Size | Std. | Zeta | Conductivity |
|---|---|---|---|---|
| | (nm) | Dev (nm) | Potential (mV) | (mS/cm) |
| PhaNP | 781.7 | 496.5 | −16.0 | 17.8 |
| PhaNP@Syn71 | 2582 | 641.2 | −8.54 | 16.8 |
| SiAuNP | NA | NA | −9.21 | 17.8 |
| Syn71 peptide | NA | NA | −7.15 | 21.0 |

LC-MS analysis on the supernatant of PhaNP@Syn71 revealed that there is no residual Syn71 peptide present in the supernatant, indicating that >99.9% of the peptide added during the synthesis process had been absorbed on the PhaNPs. Change in zeta potential when comparing PhaNPs to PhaNP@Syn71 further verified the chemisorption of Syn71 onto the PhaNP surface.

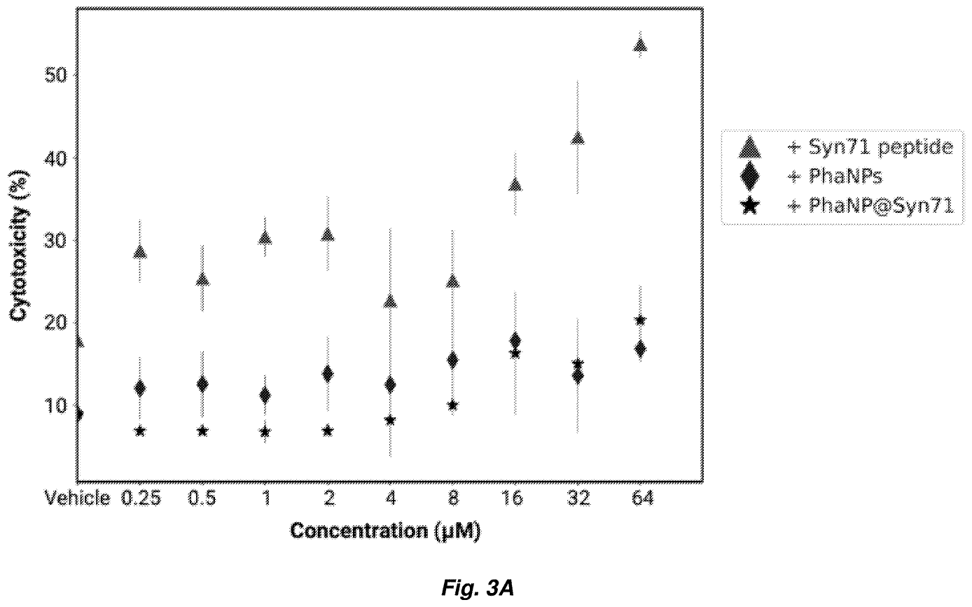

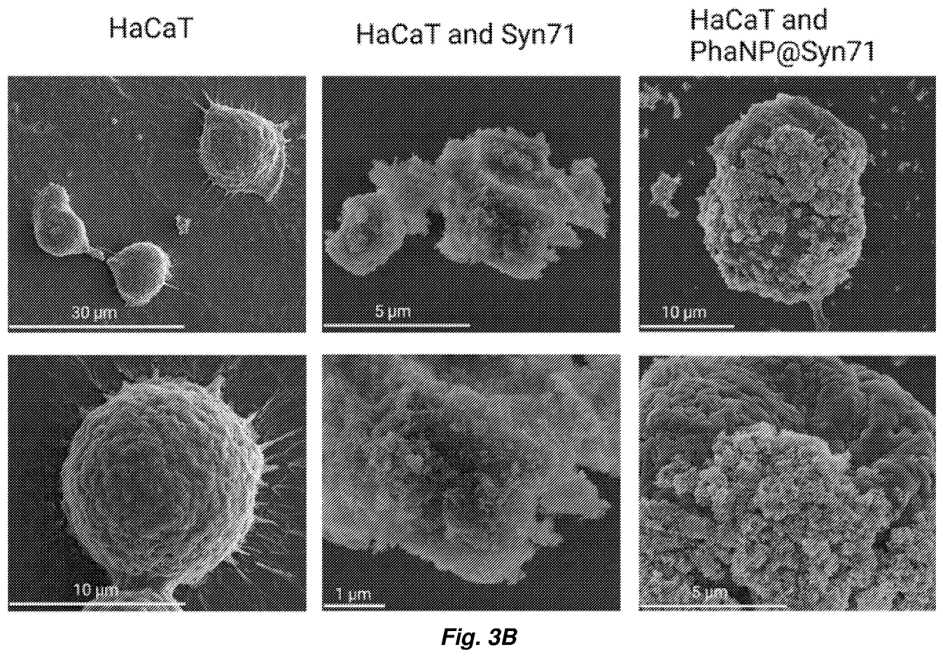

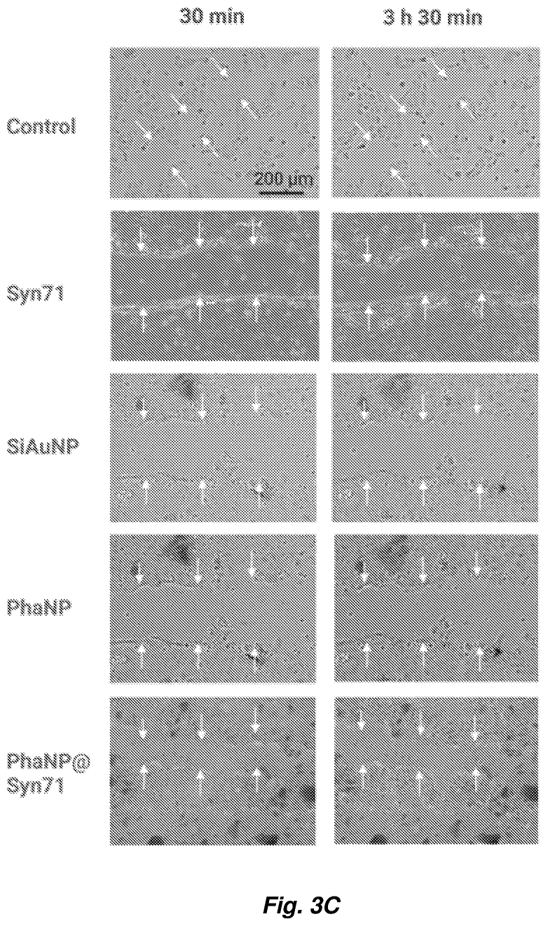

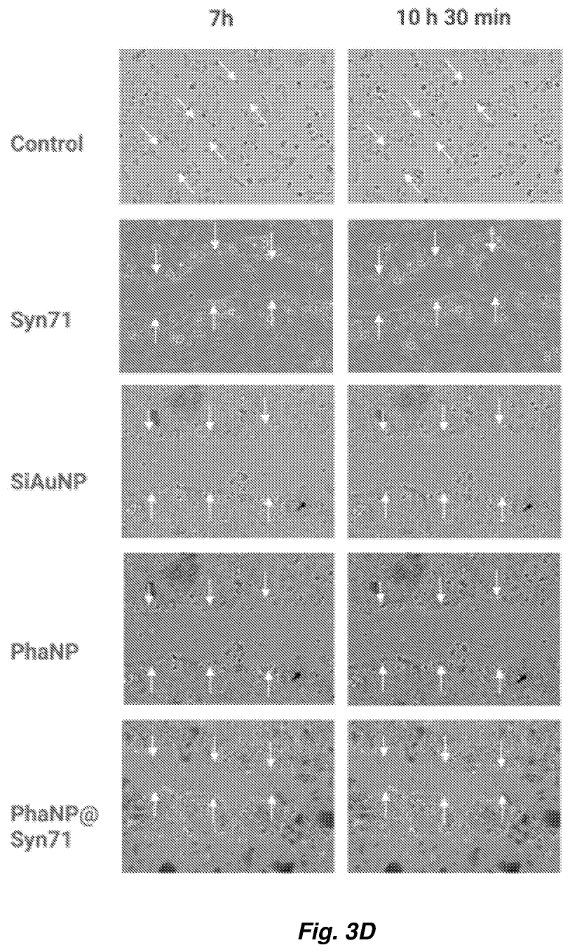

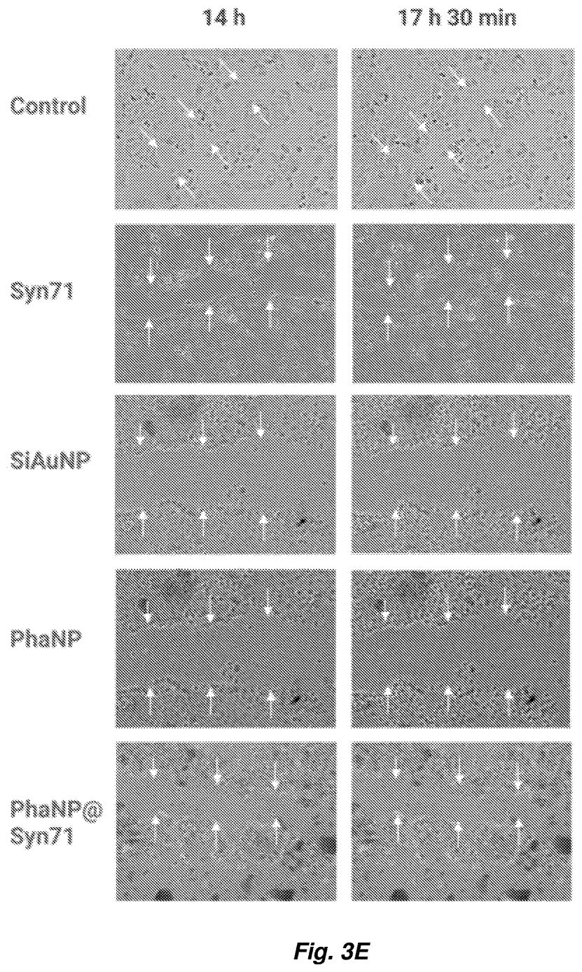

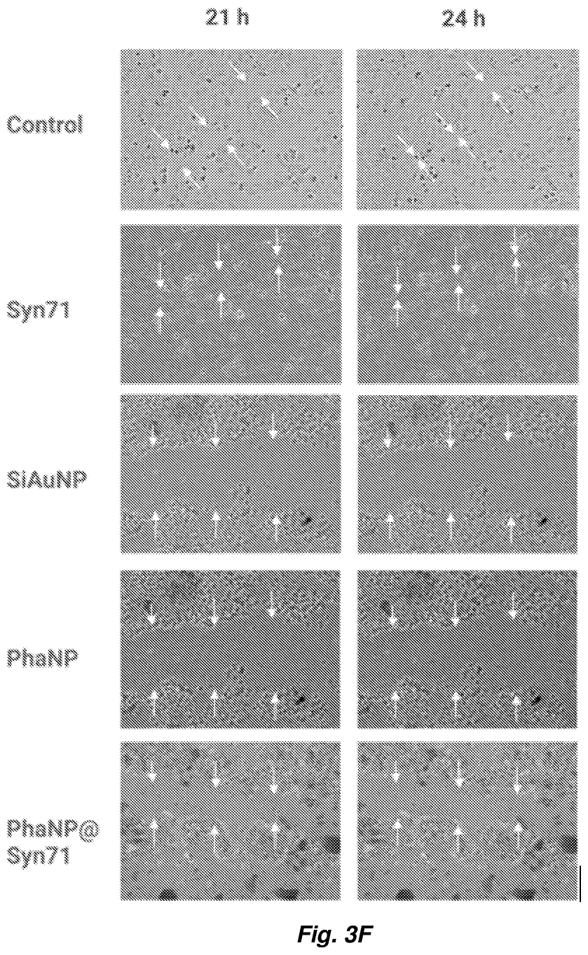

Biocompatibility with human skin cell line. Various concentrations of PhaNP@Syn71 (vehicle, 0.25 μM, 0.5 μM, 1 μM, 2 μM, 4 μM, 8 μM, 16 μM, 32 μM and 64 μM) were tested on HaCaT human skin keratinocytes (FIG. 3A) and compared against PhaNPs and free Syn71 peptide. The results showed that the PhaNP@Syn71 had significantly lower cytotoxicity than the free peptide, as well as slightly lower toxicity than PhaNPs, displaying almost no change in cytotoxicity from the vehicle up to relatively high concentrations. Syn71 peptide alone showed significantly increased cytotoxicity above 8 μM.

Silica-based materials are considered to be highly compatible with human cells with amorphous silica nanoparticles with APTES molecules similar to our designed core nanoparticles, have been shown to be biocompatible with human cells. Ag nanoparticles are also considered relatively biocompatible compared to other metallic nanoparticles, therefore they have been recommended as a good alternative for antibiotics to eradicate bacterial infections with minimal toxicity to surrounding human cells. Ag nanoparticle cytotoxicity is size-dependent with smaller particles being more toxic. Our design chose 5 nm Au/Ag nanospheres, however, the arrangement of the nanospheres on silica core ensures they are not presented as <5 nm, reducing the probability of the nanoparticles to permeabilize mammalian cells. According to Environmental Protection Agency (EPA), the chronic oral Reference Dose (RfD) is 250 μg top 750 μg per person per day, however, our PhaNP@Syn71 exhibit the maximum Ag content of 171.36 μg per ml, leaving it under the EPA established safety limits.

Several works have shown that the Minimum Inhibitory Concentration (MIC) of free antibacterial peptides is relatively high for inhibiting the growth of pathogenic bacteria, the MICs being higher than the MIC to affect human cells. Therefore, integration of the antibacterial peptides with nanoparticles in order to decrease their MIC and make them more biocompatible, has been pursued in several papers, showing that peptide integration into nanoparticles results in smaller MICs than that of the free peptide, in general. With regards to the hemolytic activity, it has been shown that the nanoparticle-peptide conjugates have decreased hemolytic activity compared to their free peptides leading to lower cytotoxicity when the nanoparticle-peptide conjugates enter the bloodstream.

Syn71 peptide exhibits dose-dependent toxicity to human cells at 16 μM and mild hemolytic activity at 8 μM. Syn71 is a synthetic, unmodified peptide derived from the active region of the AS-48 bacteriocin homologue Safencin, produced by the bacterial species Bacillus safensis. Fields et al. identified portions of this bacteriocin as scaffolds to design 25 amino acids long syn-thetic AMPs (called syn-safencin) and optimize them for charge and hydrophobicity. Our designed PhaNP@Syn71 showed significantly decreased cytotoxicity towards mammalian cells com-pared to free peptides at the same concentration. At relatively high concentrations (64 μM), PhaNP@Syn71 cytotoxicity was at 20%, while the free peptide displayed a cytotoxicity of over 50%. Very low cytotoxicity was displayed at concentrations up to 8 μM (10%), as the free peptide showed cytotoxicity higher than that of the highest concentration of PhaNP@Syn71, confirming the success of lowering the overall cytotoxicity with the developed nanoparticle-peptide conjugates.

In vitro Scratch Closure Assay. To measure the cell migration in the presence of PhaNP@Syn71, a scratch closure assay was carried out on HaCaT cells (FIG. 3C). As a control, cells in media only and cells with Syn71 pep-tide only were used, to compare against PhaNP@Syn71. A fixed concentration of 8 μM of Syn71 peptide as well as PhaNP@Syn71 was tested. The results indicated clear scratch closure in the presence of Syn71 peptide only, while the PhaNP@Syn71 did not promote any cell migration. The cell-compatible chamber slide has positively charged wells to allow the HaCaT cells to attach to the bottom of the well. However, since PhaNP@Syn71 indicated a negative zeta potential, and thus a negative charge, the nanoparticles attracted the bottom of the well instead of the cells, preventing the cells to attach to the slide. In addition, the negative charge of the PhaNP@Syn71 prevented the nanoparticles to penetrate the HaCaT cells. The cell migration assay demonstrates that the nanoparticles prevent cell movement on the slide since the cells cannot attach to the bottom of the slide well.

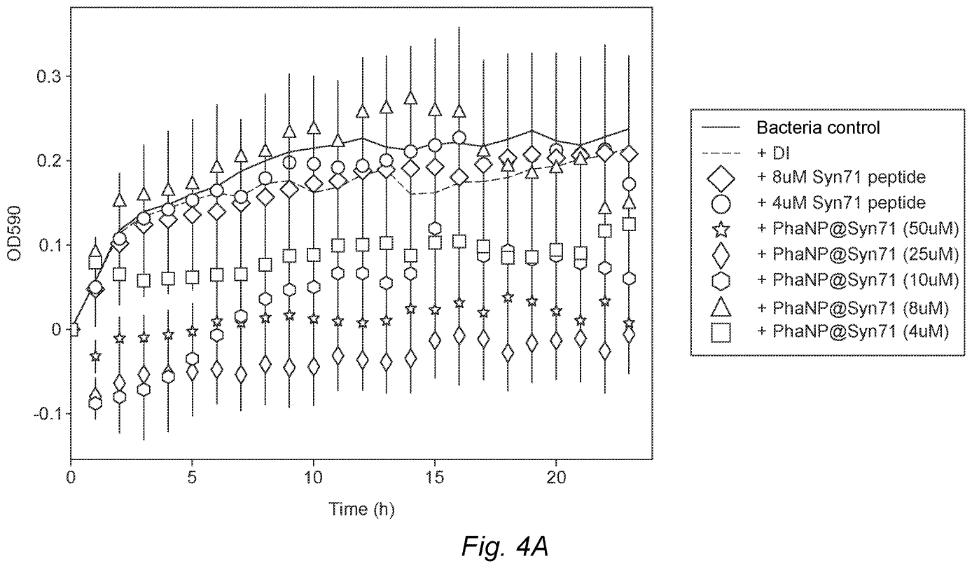

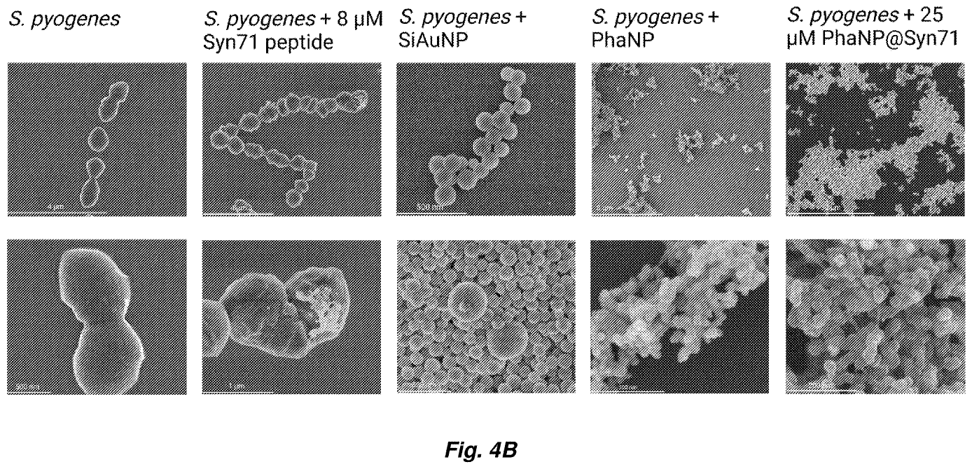

Biocidal activity of PhaNP@Syn71 against planktonic S. pyogenes. Our designed nanoparticles take advantage of the Ag antibacterial effect coupled with the antibacterial effect of the antimicrobial Syn71 peptide. By alloying Ag onto Au nanospheres, we can minimize the leaching of Ag ions, leading to lower off-target toxicity. Silver and antimicrobial peptide containing PhaNP@Syn71 have successfully shown to inhibit the bacteria growth in vitro (FIG. 4A) at concentrations between 4-50 μM. In addition, free Syn71 peptide was tested for concentrations 8 μM and 4 μM. The comparison of free Syn71 concentrations with corresponding PhaNP@Syn71 concentrations shows that at both concentrations designed PhaNP@Syn71 has higher antibacterial activity than the free peptide. Maximum 8 μM Syn71 peptide was used since the concentrations above are highly toxic to mammalian cells. Here we have shown that the S. pyogenes growth is almost fully inhibited by concentrations of 10 μM to 50 μM. PhaNP@Syn71 concentration of 4 μM also showed a significant halt on bacterial growth after initial growth. Colony Forming Units per ml (CFU/ml) further confirmed that at 50 μM concentration of PhaNP@Syn71 kills the S. pyogenes (Table 1). Although other concentrations show some live bacteria still present post-treatment, the live bacteria decreased from 3.17×10⁷ in the control group to 3.93×10⁶, with the lowest amount of live bacteria being present in the sample treated with 10 μM PhaNP@Syn71. In the growth curve, 25 μM PhaNP@Syn71 exhibits similar high inhibition as 50 μM PhaNP@Syn71, however, the CFU/ml assay shows live bacteria present post-treatment, meaning that at concentrations lower than 50 μM, the nanoparticles only inhibit the bacteria growth instead of killing. These results are relevant in displaying that our designed nanoparticle-peptide conjugates can show great potential in the inhibition of S. pyogenes growth at very low concentrations compared to a free peptide. It has been confirmed previously by other researchers that peptides conjugated on various nanoparticles display higher inhibitory activity than the peptide alone.

TABLE 1
CFU/ml assay of S. pyogenes antibacterial samples with SiAuNPs,
PhaNPs and various concentrations of PhaNP@Syn71,
compared to S. pyogenes control was carried out by
growing the bacteria collected from the growth
measurement samples on an LB agar plate at
37 °C for 14-16 h.

| ID | CFU/ml |
|---|---|
| S. pyogenes | 3.17 × 10⁷ ± 5.69 × 10⁶ |
| S. pyogenes + SiAuNP | 8.93 × 10⁶ ± 9.46 × 10⁶ |
| S. pyogenes + PhaNP | 5.13 × 10⁶ ± 5.13 × 10⁶ |
| S. pyogenes + PhaNP@Syn71 (50 uM) | 0 |
| S. pyogenes + PhaNP@Syn71 (25 uM) | 5.13 × 10⁶ ± 1.53 × 10⁶ |
| S. pyogenes + PhaNP@Syn71 (10 uM) | 3.93 × 10⁶ ± 1.36 × 10⁶ |
| S. pyogenes + PhaNP@Syn71 (8 uM) | 4.53 × 10⁶ ± 4.04 × 10⁶ |
| S. pyogenes * PhaNP@Syn71 (4 uM) | 4.27 × 10⁶ ± 2.31 × 10⁶ |

Mechanisms of antibacterial activity of PhaNP@Syn71. Several studies have previously confirmed that Ag nanoparticles conjugated with antimicrobial peptides significantly increase the overall antibacterial effect towards clinically relevant bacteria strains exhibiting antibiotic resistance. Syn71 peptide 38 is a 25 amino acids synthetic antimicrobial peptide with a cationic domain responsible for antimicrobial activity. The structure of the peptide is a random coil, adopting a helical structure in con-tact with the bacterial membrane. The positive charge of the amphiphilic Syn71 peptide allows its interaction with negatively charged bacteria membrane, leading to membrane disruption, and consecutively to cell growth inhibition or cell death. Our design poses a potential synergistic effect of Ag and antimicrobial peptide Syn71, allowing the nanoparticles to attach to the bacteria surface via positively charged peptide residues, this way disrupting the membrane and allowing the nanoparticles to enter the cell, possibly releasing Ag ions that further disrupt the intra-cellular processes.

SEM imaging was used to visualize the attachment of nanoparticles onto the bacterial cell membrane as well as the further internalization of the nanoparticles inside the bacteria cells (FIG. 4B). S. pyogenes bacteria form chain-like structures in their natural environment. The analysis of the SEM images confirmed the round coccus shape and formation of chain-like structures of untreated S. pyogenes. It has been previously shown that Syn-safencin antimicrobial peptides act via their interaction with and disruption of S. pyogenes membrane. In this case, Syn71 peptide formed microclusters on the bacteria and punctured the bacterial cell wall, confirming the previously studied effect of the peptide on the bacteria. PhaNPs with Au but without Ag and peptide showed binding to the bacteria surface, this way disrupting the bacterial chain formation, while PhaNPs with Ag but with-out the peptide exhibited significant bacterial lysis possibly due to antibacterial properties of Ag. PhaNP@Syn71 nanoparticles exhibited rapid lysis of bacteria, leading to complete destruction of the bacterial structure with no viable bacteria present in the sample and only PhaNP@Syn71 were observed on SEM images, showing that our designed nanoparticles can potentially irradicate the bacterial infections caused by S. pyogenes.

In vivo testing on mouse wound healing model. The antibacterial effect of PhaNP@Syn71 was tested in vivo on male wild type (C57bl/6) mouse (n=3-4) Streptococcus pyogenes wound healing model, experiments approved by the University of Notre Dame's IACUC committee. To introduce the infection, the S. pyogenes strain AP53S+was injected into the right flank of the mouse SubQ. On day 2 after the infection, a wound has established and treatment was begun with two treatment variants: topical application only or a combination of intravenous (IV) injection and topical application.

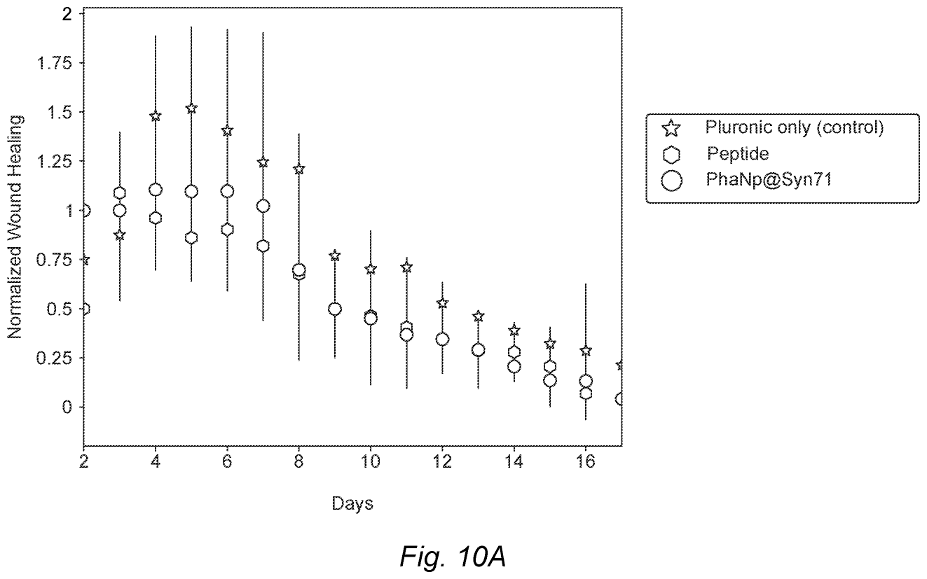

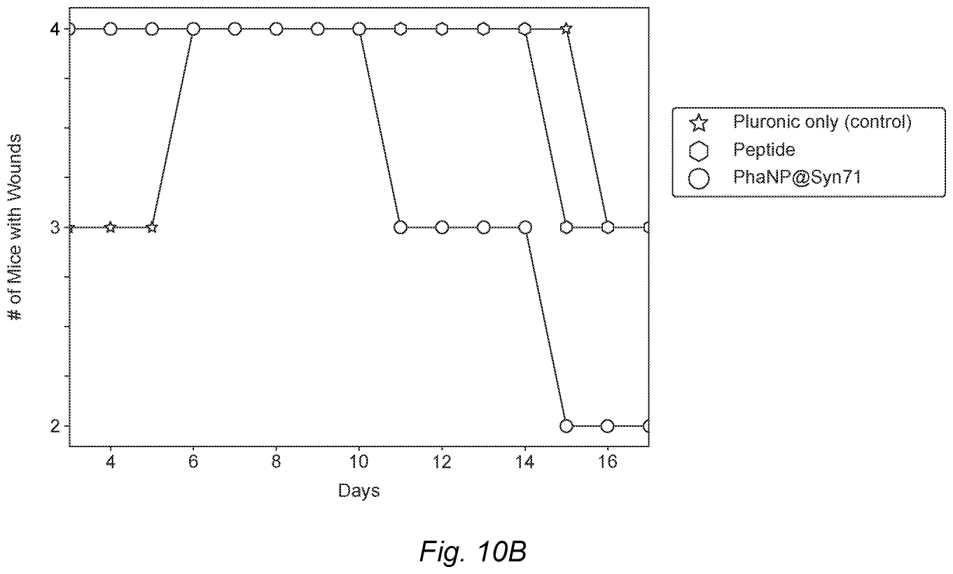

Topical only treatment of the infected wounds with PhaNP@Syn71 indicated clear stabilization of the wound size (FIG. 10), while the vehicle only and Syn71 peptide only treatment exhibited an initial increase in the wound size, indicating irritation of the wound. By Day 9 the wound had decreased to half of the initial size and fully healed by the end of the treatment course (Day 17) with PhaNP@Syn71 only topical treatment. The number of mice with wounds decreased from the initial four to three by Day 11 (FIG. 10). By Day 15, it further decreased to two mice with wounds and by the end of the treatment period (Day 17), half of the infected mice had been healed. At the end of the treatment period (Day 17), it is visible that the wound treated with PhaNP@Syn71 was fully healed, while the control and Syn71 peptide only treatments still showed a wound present on the mouse (FIG. 10).

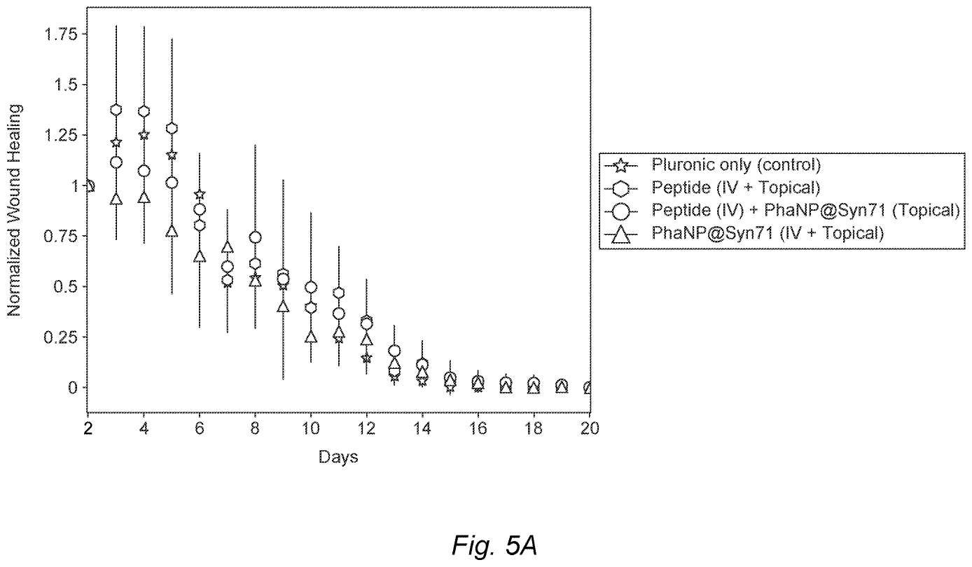

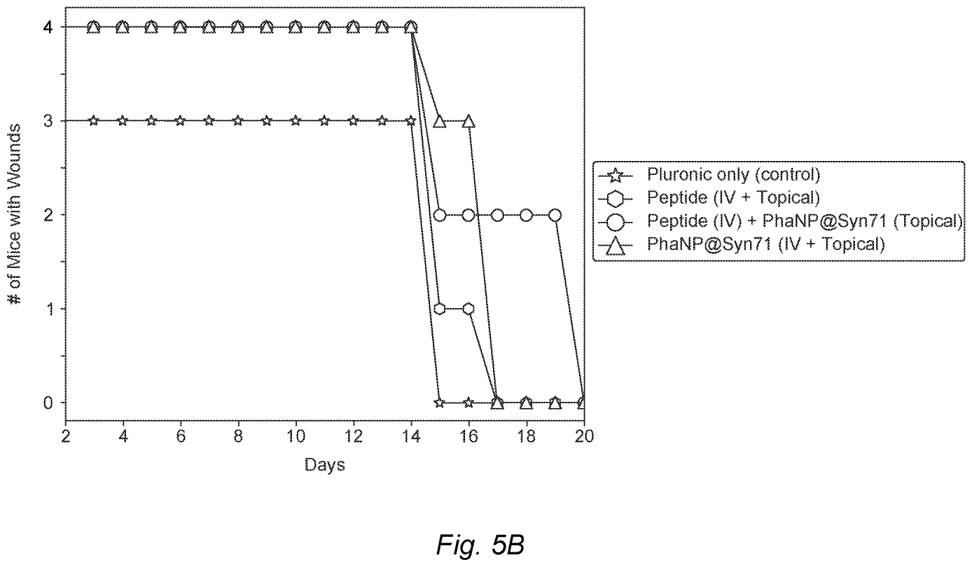

The combination of the one-time IV injection on Day 2 and further daily topical treatment was carried out using vehicle only for both IV and topical (VV), peptide only for both (PP), peptide only for IV in combination with PhaNP@Syn71 topical (NP), and PhaNP@Syn71 only for both IV and topical (NN). The combination of Syn71 peptide IV injections with PhaNP@Syn71 topical treatment resulted in an initial small increase in the wound size, similar to the vehicle only and Syn71 peptide only treatments, while PhaNP@Syn71 IV injection combined with PhaNP@Syn71 daily topical treatment of the infected wounds confirmed the initial stabilization of the wound size (FIG. 5A). The latter treatment method resulted in the wound size decreasing to half of the initial wound size by Day 8, and wounds being fully healed by Day 17. The number of mice with wounds decreased from the initial four to three by Day 15 (FIG. 5B and FIG. 5C). By Day 17 there were no mice with wounds present, indicating all mice were healed.

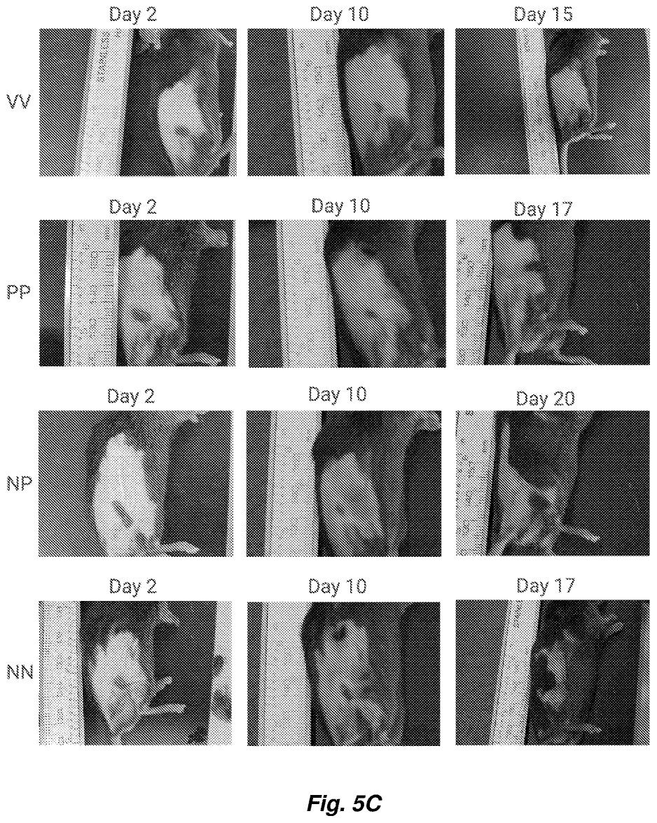

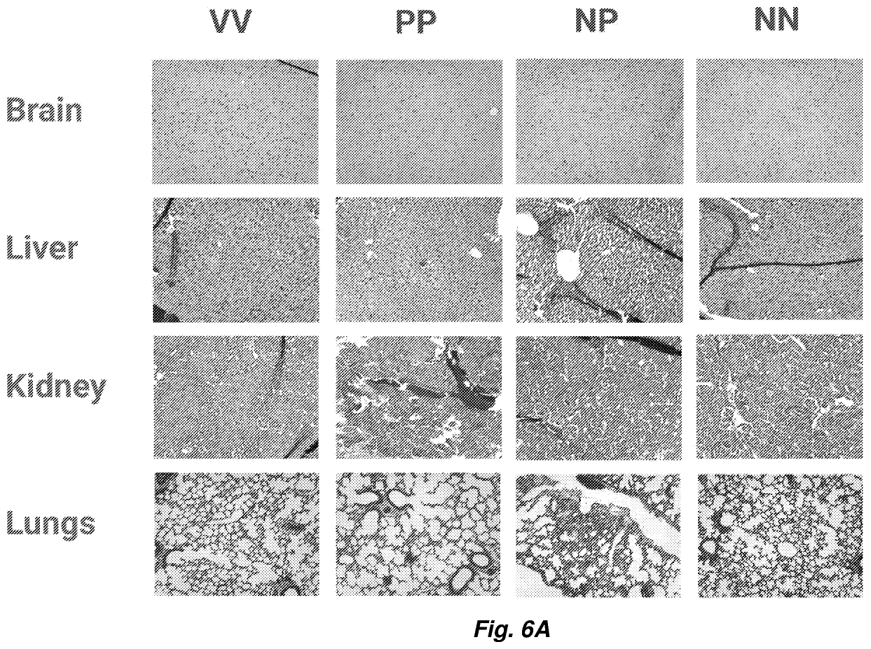

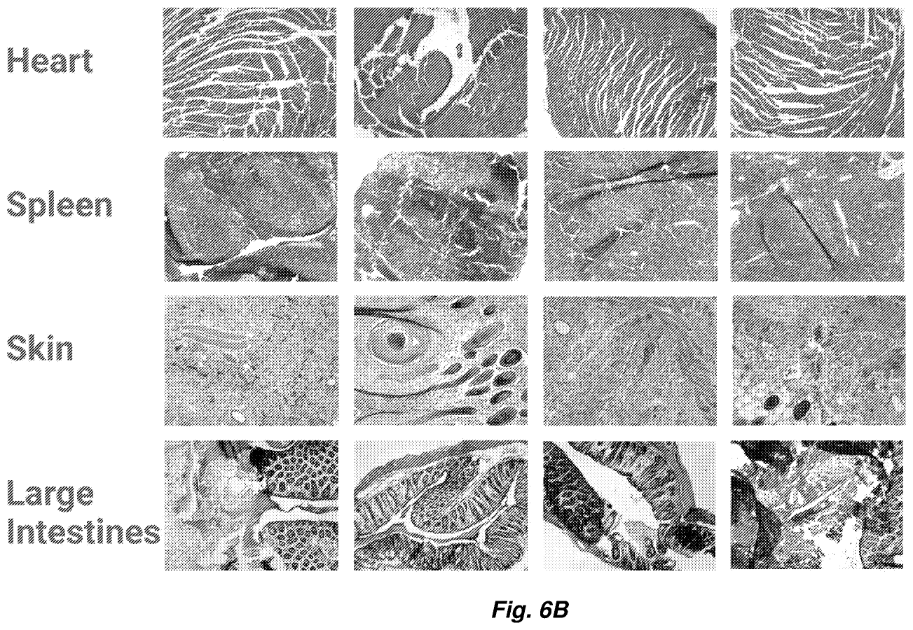

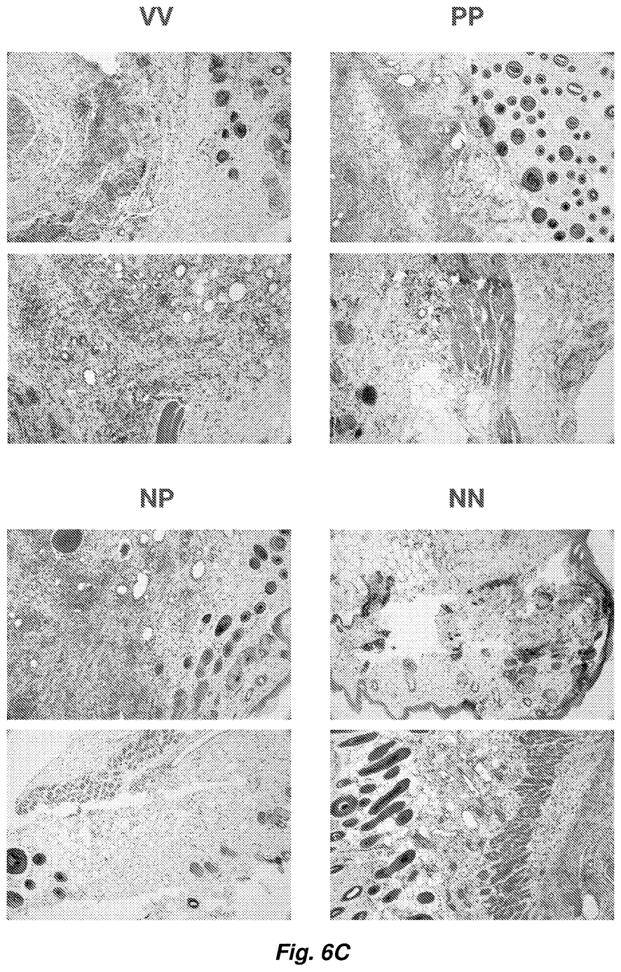

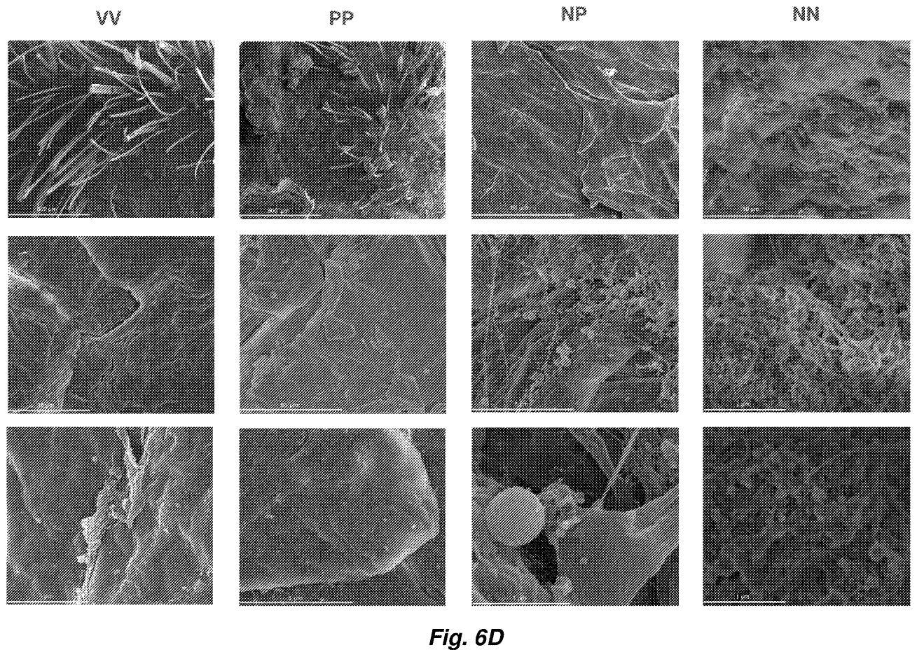

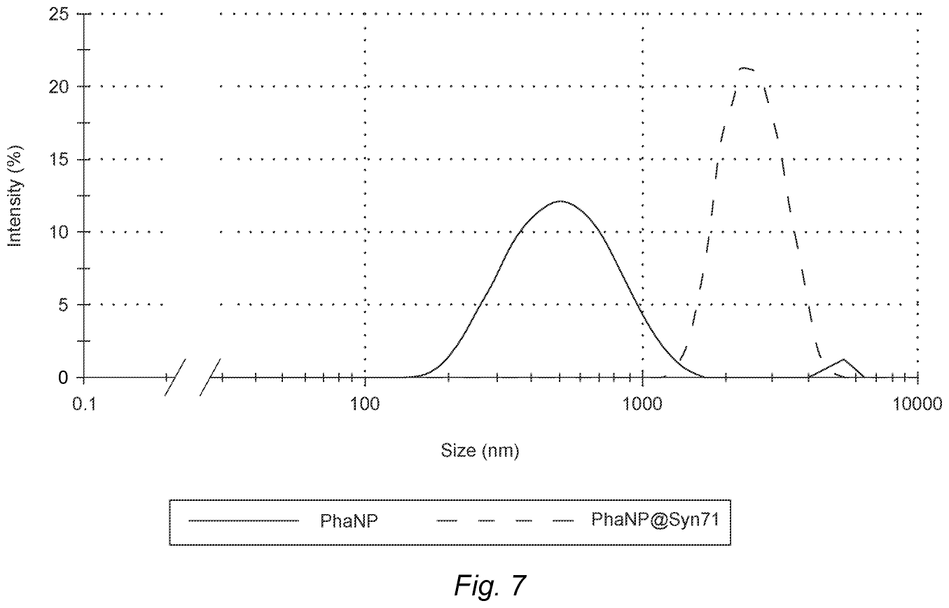

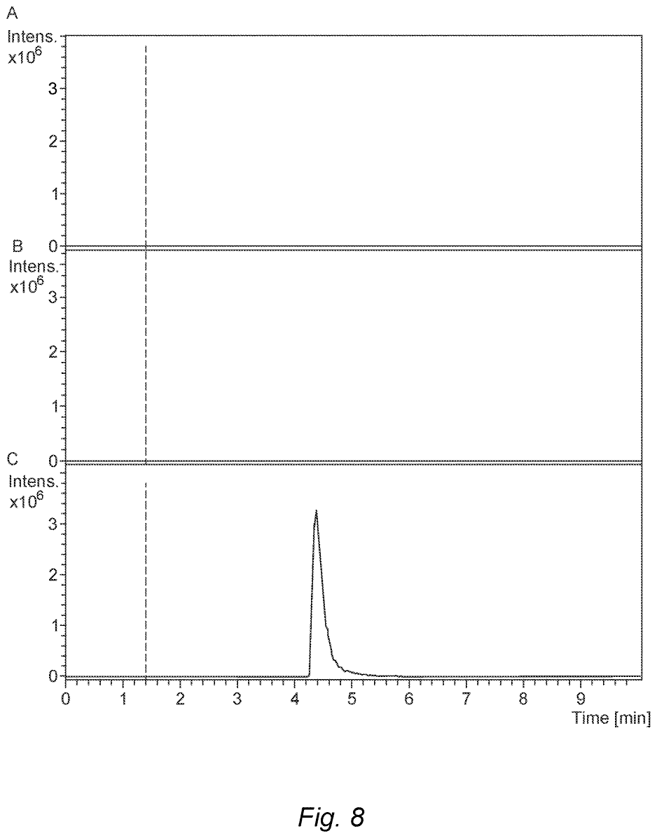

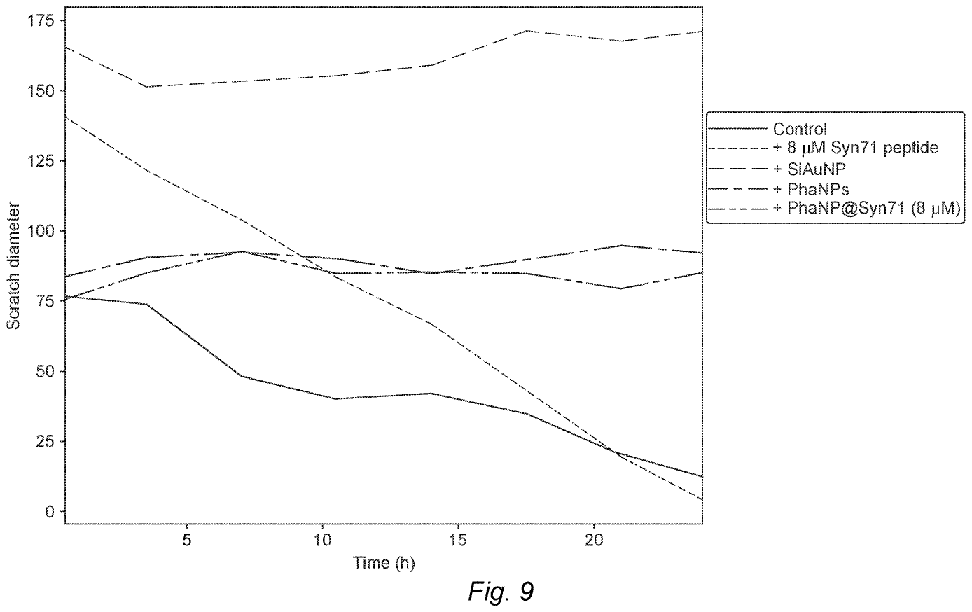

Hematoxylin and Eosin (H&E) staining of the dissected organs and skin tissue (FIG. 6A) was carried out to analyze post-treatment inflammation. There was no significant abnormal inflammation noted. Inflammation was present on skin tis-sue with the wound for all treatment groups. In mice treated with Syn71 peptide only IV injection and topical application, and the combination of Syn71 peptide IV injection and PhaNP@Syn71, there was some inflammation noted in the lungs. No additional inflammation to inflammation of the skin tissue was observed in mice treated with the combination of IV injection and topical application using PhaNP@Syn71 treatment. SEM analysis of the punctured skin tissue was carried out (FIG. 6C), confirming the histology results. Some inflammation was observed, consistent with the histology analysis, since the infected wound disrupted the tissues and created an immune response leading to inflammation. However, there were also many hair follicles present, indicating tissue regeneration.

Although there was mild inflammation in the skin tissue due to the wound infection, the histology samples of mice treated with PhaNP@Syn71 did not show increased inflammation of any of the organs, proving that our nanoparticle design is biocompatible, and does not result in the accumulation of the nanoparticles in organs. The Gram-staining of the skin tissue punctures (FIG. 6B) indicated no bacteria present in the wound after the end of the treatment period (Day 17). Therefore, PhaNP@Syn71 is fully irradicating the bacteria cells within the treatment period. In vivo results indicate that PhaNP@Syn71 topical treatment, as well as the combination of the IV injection and topical treatment, immediately stabilizes the infected wound while the control and Syn71 peptide only treatment lead to a progression in wound size before the animal's natural immunity stabilizes the wound. This proves the PhaNP@Syn71 design to be highly effective in immediately stopping wound growth.

ICP-OES analysis was carried out on dissected organs and tissues to determine the amount of the elements in nanoparticles (i.e., Si, Au and Ag) in the organs post-treatment, indicating how much nanoparticles were retained in different organs and tissues. The results were given in mg of elements per 1 g of organ/tissue. In any of the treatment options (VV, PP, NP and NN), there was no Ag present. It is visible that there is a higher amount of Si present in the lungs, spleen and fecal pellet post-treatment. It has been shown previously that silica nanoparticles mainly accumulate in the spleen, liver and lungs due to endocytosis by macrophages. They get trapped in the lungs due to passive entrapment in the endothelium opening of the liver and spleen or in pulmonary capillaries since silica nanoparticles are internalized by the reticuloendothelial system after sequestration in the liver and spleen. The accumulation of Si in fecal pellets indicates that the clearance of the nanoparticles is via the G-I tract due to their degradation in the liver and spleen. The Au particles have mainly accumulated in the lungs, spleen and heart. It was noted that there is a higher accumulation of Si and Au in the NP treatment method.

In this study, we have successfully proved that our modular design of phage-mimicking nanoparticles (PhaNPs) modified with antimicrobial peptide Syn71 (PhaNP@Syn71) has high antibacterial activity while retaining a low cytotoxic effect on mammalian cells. The biocompatibility tests on HaCaT cells exhibited significantly lower cytotoxicity to the cells than that of the corresponding concentrations of free Syn71 peptide. In addition, PhaNP@Syn71 obtained stably low cytotoxicity up to higher concentrations, with the highest concentrations of the PhaNP@Syn71 still remained lower than that of the lowest cytotoxicity of Syn71 peptide. Regarding the antibacterial effect of PhaNP@Syn71, they significantly inhibit the growth of Streptococcus pyogenes at concentrations as low as 10 μM, and completely kill the bacteria at higher concentrations. S. pyogenes does not achieve its maximum growth potential at any of the PhaNP@Syn71 concentrations tested. At equivalent concentrations, PhaNP@Syn71 displayed stronger antibacterial activity than that of its corresponding concentrations of the free Syn71 peptide. SEM analysis to study the mechanisms of action for the antibacterial effect of PhaNP@Syn71 revealed that the PhaNP@Syn71 had attached to S. pyogenes cell wall, lysing the cell and therefore inhibiting its growth and killing it since no bacterial cells were observed on SEM images when incubated with PhaNP@Syn71.

PhaNP@Syn71 showed a significant ability to improve the infection outcome in the in vivo mouse wound healing model, immediately stabilizing the S. pyogenes wound and the wound within the treatment period of 15-17 days. Further histological analysis displayed mild inflammation in skin tissues while no inflammation was observed in any of the organs, indicating that the PhaNP@Syn71 design is biocompatible. While analyzing the post-treatment accumulation of the nanoparticles, it was shown that the nanoparticles tend to accumulate in lungs, spleen and large intestines. Therefore, we could hypothesize that the post-treatment clearance of the PhaNP@Syn71 is done via the G-I tract. In conclusion, the results of this study demonstrate that our PhaNP@Syn71 design exhibits high antibacterial activity towards

S. pyogenes infections while remaining biocompatible with mammalian cells, therefore, laying a foundation for a novel antibiotic-free nanoparticle system specific to S. pyogenes infections.

### Example 2. Materials and Methods

Streptococcus pyogenes strain AP53 Covs+; lysogeny broth (LB); 200 proof ethanol (VWR); 20% w/v ammonium hydroxide (NH₄OH; BDH); tetraethyl orthosilicate (TEOS; Sigma Aldrich, 99% GC grade); 3-aminopropyltriethoxysilane (APTES; Sigma Aldrich, 99%); sodium hydroxide (NaOH; Sigma Aldrich, >97%); sodium citrate dihydrate (C₆H₅Na₃O₇·2H₂O; Sigma Aldrich, >99%); tetrakis(hydroxymethyl)phosphonium chloride (80% purity) (THPC; Sigma Aldrich, 80% in water); gold chloride (anhydrous; Sigma Aldrich); silver nitrate (AgNO₃; Sigma Aldrich, 99% ACS grade); hydroquinone (Sigma Aldrich,>99% reagent grade); deionized water (DI water); 4% buffered paraformaldehyde (VWR), Syn71 peptide, phosphate buffered saline (PBS).

Silica core synthesis. To synthesize silica (SiO₂) core nanoparticles, the sol-gel (Stöber) method of hydrolysis and condensation of TEOS was used, yielding monodispersed silica nanoparticles with a diameter of 25 nm. Into a 100 ml glass beaker with a magnetic stir bar 30 ml of ethanol, 6 ml of DI water and 1.8 ml of 20% NH₄OH were added. The mixture was stirred at a low speed of 250 rpm and 1.125 ml of TEOS was added to the mixture. The stirring speed was increased to 550 rpm at room temperature (22 °C) overnight (minimum of 12 h). The SiO₂ cores were centrifuged at 9000 rpm for 30 min at room temperature to settle the SiO₂ nanoparticles in the pellet and separate the supernatant. The nanoparticle-containing pellet was washed with 10 ml ethanol, sonicated (pulse for 20 s at 40% amplitude) and rinsed with 30 ml ethanol by centrifugation.

Silica core silanization. The silica cores were amine-functionalized by adding 25 ml ethanol, 5 ml DI water and 1 ml APTES with the silica cores into a 100 ml beaker. The solution was stirred overnight at 550 rpm at room temperature yielding SiO₂-APTES cores. The solution was centrifuged at 9000 rpm for 30 min at room temperature to separate the supernatant. The SiO₂-APTES nanoparticle pellets were washed with 10 ml ethanol, sonicated (pulse for 20 s at 40% amplitude) and rinsed with 30 ml ethanol by centrifugation. After discarding the supernatant, the pellet was resuspended in 10 ml of DI water, sonicated (pulse for 20 s at 40% amplitude) and transferred into a 100 ml beaker with additional 20 ml DI water.

Gold nanosphere synthesis. Gold nanospheres with a diameter of 5 nm were synthesized using a modified procedure of alkaline reduction (Windels et al., The ISME journal, 2019, 13, 1239-1251). Into a 250 ml beaker 43.16 ml DI water, 427 μl NaOH (1 M), 3.21 ml sodium citrate (68 mM) and 1.07 ml THPC (85 mM) were added. The solution was stirred at low speed (250 rpm) for at least 10 min at room temperature, followed by the addition of 2.14 ml of gold chloride (25 mM). The solution was stirred at 550 rpm at room temperature overnight in dark.

Silver coating of gold nanospheres. Silver is alloyed onto gold nanospheres previously conjugated onto SiO₂ nanoparticles using the reduction reaction by hydroquinone (Gentry et al., Langmuir, 2009, 25, 2613-2621.). 30 ml of SiO₂@Au nanoparticle solution was mixed with 7.2 ml 10 mM AgNO₃ and 5.76 ml 10 mM freshly prepared hydroquinone. The mixture was stirred at 500 rpm overnight in dark, followed by halting the silver coating reaction by centrifugation and discarding the supernatant. SiO₂@Au@Ag (PhaNPs) pellet was washed with 30 ml DI water two times, sonicated (pulse for 20 s at 40% amplitude) and rinsed by centrifugation at 9000 rpm for 15 min at room temperature.

Antimicrobial peptide chemisorption onto PhaNPs. MC1-2, Syn20 and Syn71 are cysteine-terminated antibacterial peptides. Syn20 and Syn71 peptides were synthesized by Fields et al., The Journal of antibiotics, 2018, 71, 592-600 and Ross et al., Frontiers in Microbiology 11, 589666, respectively. MC1-2 peptide was synthesized by Dong et al., Scientific reports 2017, 7 (1), 1-13. 5 mg/ml MC1-2, 5 mg/ml Syn20 and 4 mg/ml Syn71 peptide stocks were prepared in 1×PBS. PhaNP pellet was resuspended in 30 ml 1× PBS and added into a 100 ml glass beaker with 1 ml of MC1-2 (5 mg/ml), 1 ml Syn20 (5 mg/ml) or 1 ml Syn71 peptide (4 mg/ml). The solution was stirred at 500 rpm at room temperature overnight in the dark for at least 36 h, allowing maximal chemisorption of the peptide onto the PhaNPs, yielding PhaNP@MC1-2, PhaNP@Syn20 and PhaNP@Syn71. PhaNP@Peptide solution was centrifuged at 9000 rpm at room temperature for 15 min, the supernatant was saved and stored in a −20 °C freezer for further Liquid Chromatography Mass Spectroscopy (LC-MS) analysis. The nanoparticle pellet was washed in 15 ml 1× PBS, sonicated (pulse for 10 s at 30% amplitude) and rinsed by centrifugation. The second supernatant was saved and stored in the freezer as the first one.

TEM imaging. SiO₂ core and PhaNP@Syn71 nanoparticle samples were pre-pared for TEM analysis by drop coating the dispersed sample onto a 300-mesh copper grid coated with amorphous carbon film. TEM images of the SiO2 core and PhaNP@Syn71 nanoparticles were obtained on a JEOL 2011 microscope at an acceleration voltage of 200 kV. Further image analysis was carried out using ImageJ software. Diameters of 102 SiO2 core nanoparticles and 102 PhaNP@Syn71 nanoparticles were measured from several TEM images selected and the average diameters of both samples were calculated.

SEM/EDXS analysis. To analyze the nanoparticle elemental content and confirm the presence of silica, gold and silver, SEM in conjugation with (EDXS) analysis was carried out on Thermo Scientific Prisma E-SEM at an acceleration voltage of 15 kV, current of 70 pA and magnification of 100,000×. A drop of resuspended PhaNP sample was placed onto an aluminum SEM stub and dried under vacuum. An EDXS map was created to scan over a site of PhaNPs on the stub, with a count rate of 993 cps.

Inductively coupled plasma-optical emission spectroscopy (ICP-OES) for nanoparticle elemental quantification. ICP-OES was performed on PerkinElmer Optima 8000 to quantify the elemental distribution of silica (Si), gold (Au) and silver (Ag). 0, 0.2, 2, 4, and 10 ppm Si, Au and Ag standards were pre-pared from commercially available 1000 ppm Si, Au and Ag (II) standard solutions and dissolved in 5% Aqua Regia. 200 μl of the SiNP, SiAuNP and PhaNP samples were dissolved overnight in 500 μl of 100% Aqua Regia (3:1 hydrochloric acid: nitric acid), followed by the addition of 9.5 ml DI water to get the final concentration of 5% Aqua Regia. Emission spectra for Si, Au and Ag were collected at 251.611 nm, 267.595 nm and 328.068 nm, respectively. Emission spectra for yttrium (Y), an internal standard, were collected at 371.029 nm.

Liquid Chromatography Mass Spectrometry (LC-MS) analysis. The collected supernatants from the peptide modification step in the PhaNP@Peptide synthesis were stored in a freezer (−20 °C) to be analyzed on Liquid Chromatography Mass Spectrometry (LC-MS) and quantify the amount of each peptide (MC1-2, Syn20, Syn71) absorbed on PhaNP@Peptide by back-calculating it from the peptide residues in the supernatant. The LC-MS instrument consisted of a Dionex Ultimate 3000 Rapid Separation UPLC system equipped with a Dionex Ultimate 3000 autosampler and a Dionex Ultimate 3000 photodiode array detector coupled with a Bruker MicrOTOF-Q II quadrupole time-of-flight hybrid mass spectrometer using Hystar 3.2 software. The parameters of the positive ion mode that the Bruker electrospray ionization source was operated were predefined as follows: end plate offset voltage =−500 V, capillary voltage =2000 V, and nitrogen as both a nebulizer (4 bar) and dry gas (8 L/min flow rate at 180 °C temperature). Mass spectra were accumulated over the mass range 400-3000 Da. The samples were analyzed on a Dionex Acclaim™ RSLC 120 C₈ column (2.2 μM, 120 Å, 2.1 mm i.d.×100 mm) with a mobile phase (A=0.1% formic acid in water; B=0.1% formic acid in acetonitrile) gradient consisting of elution at 0.4 mL/min with 90% A/10% B for 1.5 min, followed by a 6.9-min linear gradient to 5% A/95% B, an 0.1-min linear gradient to 90% A/10% B, and then 90% A/10% B for 1.5 min. For the quantification, peak areas from extracted ion chromatograms (EICs) of corresponding m/z values were used.

Dynamic Light Scattering (DLS). The size distributions of the plain and peptide-bearing nanoparticles were measured using a Malvern Zetasizer Nano-ZS (Model ZEN3600) instrument by performing DLS. Each DLS sample was prepared by diluting 100 μL of the nanoparticle stock solution with 900 μL of 1× phosphate-buffered saline (PBS). The resulting dilutions were carefully pipetted into 1 mL polystyrene cuvettes (Malvern part number DTS0012) to prevent bubble formation. The cuvettes were loaded into the warmed-up Zetasizer one at a time by orienting the arrow onto the cuvette to the arrow on the instrument and closing the device's lid. The Malvern Zetasizer software was used to take the size measurements. The program uses the absorption and refractive index of the nanoparticle's silica core material (Ri =1.459, A=0.035) and the refractive index, dielectric constant, and viscosity of the PBS dispersant ((Ri)PBS=1.330, K=79, μ=0.8882 cP) as well as its internal measurements to determine the size distribution of the nanoparticles. The instrument reports three size distributions that correspond to the three trials the instrument automatically performs when run. The software determines the sizes reported for each trial by averaging eleven different measurements taken during each trial.

Electrophoretic Light Scattering (ELS). The zeta potential values of the plain and peptide bearing nanoparticles were also found with the Malvern Zetasizer Nano-ZS (Model ZEN3600) instrument by performing ELS. Each ELS sample was prepared by diluting 100 μL of the nanoparticle stock solution with 900 μL of 1× phosphate-buffered saline (PBS), as was done for the DLS samples. These dilutions were carefully pipetted into folded capillary zeta cells (Malvern part number DTS1070) to prevent bubble formation. These cells were loaded into the warmed-up Zetasizer one at a time by matching the arrow onto the cell to the arrow on the instrument and closing the device's lid. The Malvern Zetasizer Nano-ZS software was used to take the zeta potential measurements. The program uses the absorption and refractive index of the silica core material ((Ri)Si=1.459, A=0.035) and the refractive index, dielectric constant, and viscosity of the PBS dispersant ((Ri)PBS=1.330, K=79, μ=0.8882 cP) as well as its internal measurements to determine the zeta potential. The instrument reports three zeta potential measurements that correspond to the three trials the instrument automatically performs when run. The software displays the average zeta potential found over the 100 measurements the device takes during each trial.

Cytocompatibility testing. To test the cytocompatibility of the PhaNP@Syn71, HaCaT human epithelial keratinocytes were used. The cells were cultured and maintained in Dulbecco's Modified Eagle's Medium (DMEM) supplemented with 10% heat-inactivated fetal bovine serum (FBS) at 37 °C with 5% CO₂ in 100 mm culture dishes. First, HaCaT cells were grown to 75-80% confluency in 24-well tissue culture plates supplemented with DMEM. Before adding the samples (PhaNP@Syn71, PhaNP, Syn71), DMEM was aspirated from the wells and the cells were washed with 1×PBS. The PBS was aspirated and the sample solutions in DMEM were added to the wells with washed HaCaT cells. The cells were incubated at 37 °C with 5% CO₂ for 16 h, after which, DMEM was aspirated and cells washed with 1×PBS to remove free nanoparticles. To determine the cytotoxicity of various concentrations of PhaNP@Syn71 as well as PhaNPs and Syn71 peptide alone, ethidium homodimer cell death assay 30 was used. Four μM ethidium homodimer in 1×PBS was added to HaCaT cells and incubated at 37 °C for 30 min. The plate reader was set to 528 nm excitation and 617 nm emission with a cut-off value of 590 nm to determine the level of fluorescence. To determine the percentage of dead cells, all cells were permeabilized by the addition of 0.1% (w/v) saponin to each well after the initial reading, and incubated at room temperature for 20 min, followed by shaking the plate on an orbital shaker. The plate reader was used in the same settings as before to measure the fluorescence. Percent membrane permeabilization was calculated by dividing the fluorescence values with intact cells by the fluorescence values after cell disruption by saponification. Each treatment condition was performed in triplicate, with the average cell viability (as a percent of the control) plus the standard deviation of all conditions plotted together for comparison.

In vitro Scratch Closure Assay. In vitro Scratch Closure Assay was carried out on HaCaT human epithelial keratinocytes by using a modified protocol by Liang et al. Nature protocols, 2007, 2, 329-333. The cells were cultured and maintained in Dulbecco's Modified Eagle's medium (DMEM) supplemented with 10% heat-inactivated fetal bovine serum (FBS) at 37 °C with 5% CO₂ in 100 mm culture dishes until confluency reached 80-90%. HaCaT cells were prepared by washing the cells with 1×PBS. Trypsin was added and cells were incubated at 37 °C for 5 min followed by stopping trypsinization by adding DMEM. Cells were centrifuged at 1200 rpm for 12 min and the pellet was resuspended in DMEM. An 8-well chamber slide was used and 50,000 HaCaT cells were added to each well. Corresponding volumes of free Syn71 pep-tide (8 μM), SiAuNPs, SiAuAgNPs and PhaNP@Syn71 (4 μM and 8 μM) were added. Enough DMEM media was added to reach the final volume of 300 μl per well. The cells were allowed to attach to the well bottom for 26 h at 37 °C, followed by creating the scratch by using a sterile pipette tip to scrape the cell monolayer in a straight line. The chamber slide was live imaged under the microscope at 37 °C for 24 h.

Biocidal activity against planktonic bacteria. The antibacterial effect of various concentrations of PhaNP@MC1-2, PhaNP@Syn20 and PhaNP@Syn71 were tested against S. aureus USA300, P. aeruginosa FRD1, A. baumannii, K. pneumoniae and S. pyogenes. As controls, Si@Au nanoparticles, PhaNPs without peptide modification and each free peptide were tested on the same bacteria.

Bacteria culture. S. aureus USA300, P. aeruginosa FRD1, A. baumannii and K. pneumoniae were cultured on a Luria Broth (LB) agar plate. The stocks for A. baumannii and K. pneumoniae were created from KWIK-STIK lyophilized bacteria pellets. All stocks were stored at −80 °C prior to plating them on the LB agar plate. Plates were incubated for 16-18 h at 37 °C. S. pyogenes blood agar plate were kept in a fridge at +4 °C for up to two weeks. A single colony of each bacteria strain was picked from the plate and grown in liquid LB media for 12 h at 37 °C under continuous shaking (300 rpm) to adapt the bacteria to a planktonic lifestyle. The inoculate was then used as a pre-culture for the growth curve measurements on a plate reader.

Growth curve measurements. At least four replicates of each sample and control were run on Perkin Elmer Victor 3 1420 Multilabel Counter Plate Reader. As a background and positive control, pure LB media and the bacteria growing in LB media only were used. The tests were premixed in a 2 ml microcentrifuge tube, containing a total of 2 ml of solution, including 30 μl of bacteria inoculum and precalculated volumes of nanoparticle samples as well as Syn71 peptide alone. In addition, background solutions for each nanoparticle sample and concentration without bacteria were prepared. The solutions were mixed thoroughly and 200 μl of the solution was pipetted into each well of a sterile 96-well plate and incubated under continuous shaking at 37 °C for 24 h with OD590 measurements taken every 10 min. The resulting growth curves were corrected with the LB media background as well as the nanoparticle backgrounds.

CFU/ml assay. The inoculum from a 96-well plate after growth curve measurements were collected for each sample from 3 wells of 200 μl (bacteria alone, bacteria with SiAu nanoparticles, PhaNPs and all concentrations of PhaNP@Syn71). Serial dilutions were carried out by adding 100 μl sample inoculum to 900 μl LB media. Dilutions of 10-3, 10-4, 10-5 and 10-6 were plated for CFU/ml assay. Blood agar plates were used to plate at least 3 replicates of 10 μl of each sample. The plates were incubated at 37 °C for 16-18 h. CFU/ml was calculated according to Equation 1.

$$\frac{CFU}{ml} = \frac{counted CFU}{total inoculum volume} \times \frac{1000 μ l}{1 ml} \times \frac{1}{dilution factor} \qquad ( 1 )$$

Bacteria imaging with Electron Microscopy (EM). Scanning Electron Microscopy (SEM) was carried out on Magellan 400 XHR. Bacteria samples were collected after the growth measurements, taking 400 μl of each bacteria sample. Round 12 mm coverslips were prepared by dipping them into 0.1% (w/v) poly-L-lysine for 15 min and air drying overnight. As a primary fixative, 2% glutaraldehyde was prepared in 0.1 M sodium cacodylate buffer (pH 7.5). As a secondary fixative, 1%0.0 was used. The bacteria cells were centrifuged at 1200 rpm for 10 min and the supernatant was removed. The samples were washed three times in 1×PBS by centrifugation. After the final centrifugation, the cells were resuspended in 100 μl 1× PBS, and 50 μl of each sample was applied in duplicates onto separate poly-L-lysine coverslips prepared earlier and incubated for 15 min at room temperature. 500 μl of glutaraldehyde fixative was added onto samples on coverslips and incubated for 1 h after which the coverslips were washed three times with sodium cacodylate buffer. The coverslips were further incubated with the 0.0 fixative for 1 h, rinsed three times with sodium cacodylate buffer, two times with DI water and finally dehydrated with ethanol followed by replacing ethanol from the final dehydration step with liquid CO₂ in a critical point dryer. Each coverslip was applied on the SEM stub and coated with iridium for imaging.

In vivo wound healing studies. Male wild type (C57bl/6) mice (age 6-10 weeks) were prepared with N/Air on the right flank. 100 μL of Streptococcus pyogenes strain AP53S+was injected SubQ with a concentration of 1-3×10 8 CFU/mouse under isoflurane anesthesia. After the wound was established, on day 2, daily measurements and a 100 μL dose of either vehicle, peptide, or nanoparticles with peptide were given topically. The topical dose w.r.t peptide was 150 μM, suspended in 20% w/v Pluronic® F-127. After 15 days, the mice were sacrificed. In the second experiment, all the previous steps were followed. The intravenous (IV) dose w.r.t peptide was 100 μM, suspended in 1×PBS. A retro-orbital injection of 100 μL was done with either vehicle, peptide or nanoparticles on day 2 when topical treatment was begun. Wound sizes were recorded daily. The mice were allowed to fully heal before anaesthetizing with a rodent cocktail (9 parts ketamine (100 mg/mL)+9 parts xylazine (20 mg/mL)+3 parts acepromazine (10 mg/mL)+79 parts saline), (body weight×10)-50 μL=μL/mouse and col-lection of blood and tissues. All procedures were approved by the University of Notre Dame's IACUC committee. The wound size data was normalized for the analysis based on the size on Day 1. The normalized wound sizes were then averaged for each day and standard deviations were calculated. After the mice were sacrificed, they were dissected, tissues and organs collected and fixed in 4% buffered para-formaldehyde. The histology samples were prepared for H&E staining and Gram-staining. Stained samples were imaged under Swift Binocular Compound Microscope SW350B with SWIFT 1.3 Megapixel Digital Camera for Micro-scope Eyepiece Mount Windows/Mac to record the images.

Inductively coupled plasma-optical emission spectroscopy (ICP-OES) for quantification of nanoparticles present in mice organs post-treatment. Using a scalpel, a longitudinal piece of each organ was harvested and weighed. Samples weighed between 0.26 g and 0.01 g. After weighing, the samples were added to a centrifuge tube. 500 μL of Aqua Regia (1:3 Nitric Acid: Hydrochloric Acid) was added to begin digestion. A glass rod was used to break down the sample into small pieces. The samples were allowed to digest for 1-7 days. Samples were diluted to 5% Aqua Regia with purified DI water. Samples were mixed by vortexing and then filtered through a 1.0 μM Nylon syringe filter before testing.

The samples were run on a Perkins Elmer Avio 200 using a 5-point standard curve created from Si, Ag, and Au standards (LabChem). The instrument was blanked using a 5% Aqua Regia and purified DI water solution. The concentrations of elements in each sample were related to the weight of digested samples.

### Example 3. Peptide-Conjugated Phage-Mimicking Nanoparticles Exhibit Size-Dependent, Broad-Spectrum Antibacterial Activity

Nanoparticle characterization. Current work is based on phage-mimicking nanoparticles (PhaNPs) resembling naturally occurring bacteriophages, and that have previously been tested on various antibiotic-resistant bacteria strains, exhibiting high (>99.9%) inhibition in bacteria growth. Here, we synthesized two different-sized PhaNPs (20 nm and 95 nm), further modified with three different antimicrobial peptides (MC1-2, Syn20 and Syn71). To minimize the leaching of Ag ions and off-target toxicity, we have opted for alloying Ag onto Au nanospheres.

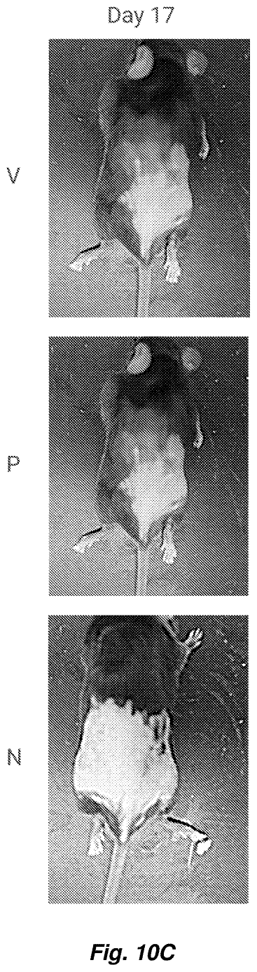

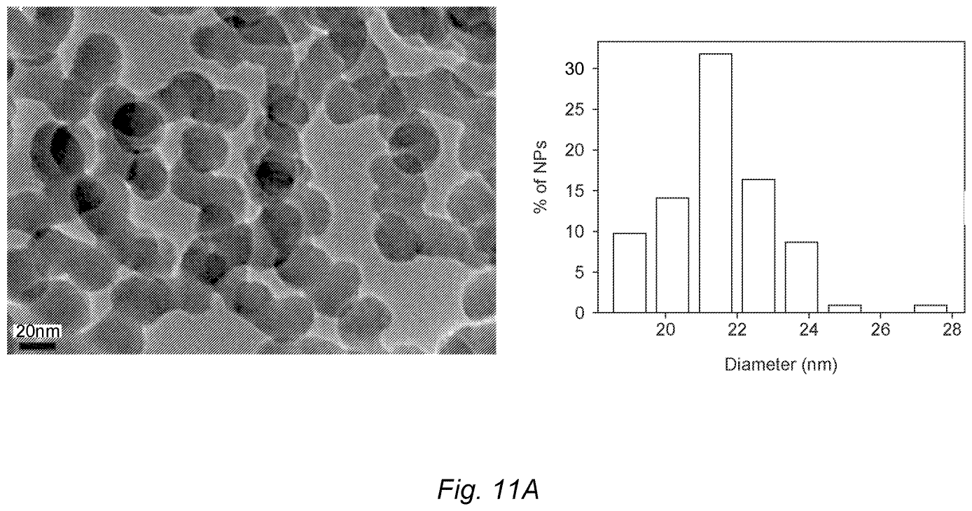

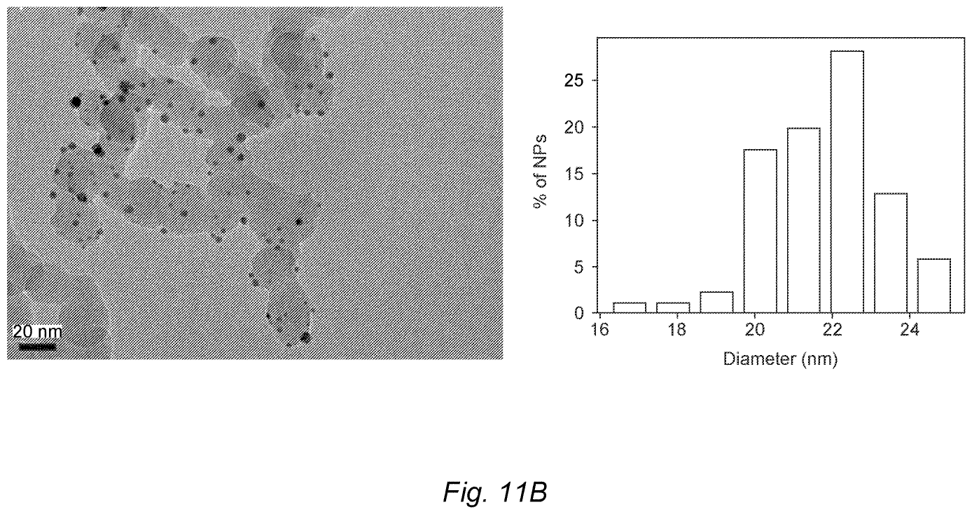

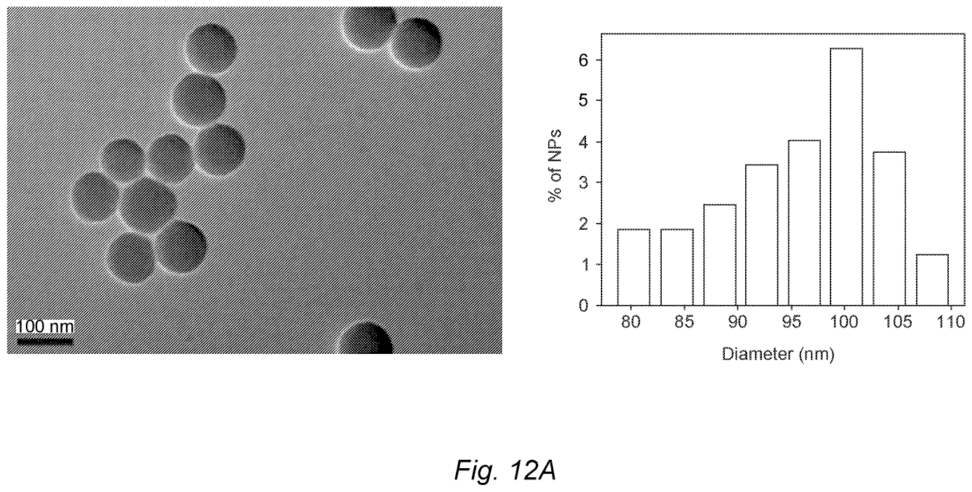

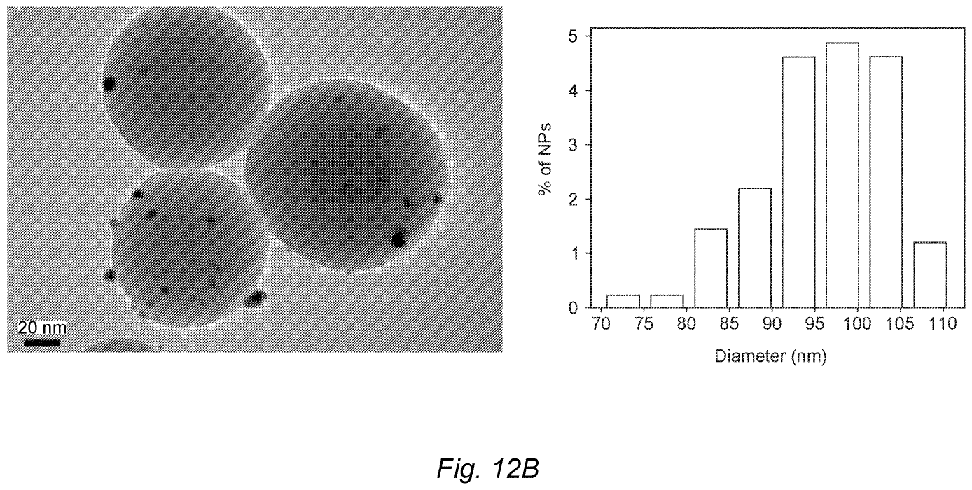

TEM imaging showed that the average diameter of the smaller SiO₂ core nanoparticles was 21.57±1.63 nm and the diameter of PhaNPs after the synthesis was 21.76±1.60 nm (FIG. 11). As for the bigger nanoparticles, the average diameter of SiO₂ core nanoparticles was 95.79±7.43 nm while the diameter of PhaNPs was 96.01±7.76 nm (FIG. 12). These results confirm that the synthesis process of adding gold-silver (Au-Ag) nanospheres onto the SiO₂ core does not alter the base diameter of the nanoparticles. TEM analysis also confirmed the presence of Au-Ag nanospheres on SiO₂ core nanoparticles, indicating the synthesis process was successful. In addition, EDXS analysis was carried out to further confirm the spheres on SiO₂ core were Au and Ag. PhaNP@Peptides couple the Ag antibacterial effect with the antibacterial effect of various antimicrobial peptides chemisorbed onto the PhaNP surface. The antimicrobial peptides MC1-2, Syn20 and Syn71 were further chemisorbed onto the PhaNPs. MC1-2 peptide was derived by Dong et al., Scientific reports 2017, 7 (1), 1-13, by extracting antimicrobial peptide Chensinin-1 from the skin secretion of Chinese frog Rana chensinensis, acting against different microorganisms to protect the naked skin from infections. These peptides are known to permeate and destroy bacterial plasma membrane and inactivate the intracellular target, preventing the target organism from developing resistance against the peptide.

MC1-2 was derived from purified Chensinin-1 with the replacement of glycine with tryptophan (Table 2) and the removal of the histidine residues. Syn20 peptide, developed by Fields, is derived from AS-48 bacteriocin produced by Enterococcus spp. Portions of this bacteriocin were identified as scaffolds to design 25 amino acids long synthetic AMPs (called syn-safencin), further optimized for charge and hydrophobicity by replacing glycine in the original syn-safencin sequence with tryptophan and glutamic acid with lysine (Table 3). Syn71 (syn-larvacin) is derived from an AS-48-like bacteriocin ribosomally produced by Paenibacillus larvae. From the previously identified active region, a 25-mer larvacin scaffold was chosen, which was further modified by lysine and tryptophan substitutions for aliphatic and short-chain amino acids to increase lipid membrane affinity and penetration, respectively (Table 4). Hydrophobic amino acids within the helical wheel were inverted to increase the amphipathicity, leading to binding to and disruption of the lipid membrane. Tryptophan replacements are known to facilitate the anchoring of the peptide onto the cell bilayer surface through the interaction with the interfacial region of the cell membrane, while lysine substitution increases the positive charge and localizes it to one side of the peptide, leading to interactions with lipids, disruption of the lipid organization of the bacterial cell wall and increased permeability of the bacterial cell.

TABLE 2
Sequence of MC1-2 peptide 27 compared to the
naturally occurring Chensinin-1 and modified
MC1-1 peptide. The replacements of amino
acids are indicated in bold. MC1-2 is
identical to MC1-1 with histidine residues
removed.

| Peptide Variant | Sequence |
|---|---|
| Chensinin-1 | SAVGRHGRRFGLRKHRKH |
| | (SEQ ID NO: 4) |
| MC1-1 | SAVWRHWRRFWLRKHRKH |
| | (SEQ ID NO: 5) |
| MC1-2 | SAVWRHWRRFWLRKHR |
| | (SEQ ID NO: 6) |

TABLE 3
Sequence of Syn20 peptide 25 compared
to enterocin AS-48 homologue Syn-
safencin. The replacements of amino
acids are indicated in bold.

| Peptide Variant | Sequence |
|---|---|
| Syn-safencin | AGKETIRQYLKNEIKKKGRKAVIAW |
| | (SEQ ID NO: 7) |
| Syn20 | AWKKTIRQYLKNKIKKKGRKAVIAW |
| | (SEQ ID NO: 8) |

TABLE 4
Sequence of Syn71 peptide 26 compared to
enterocin AS-48-like homologue Syn-larvacin.
The replacements of amino acids
are indicated in bold.

| Peptide Variant | Sequence |
|---|---|
| Syn-larvacin-1 | AGKETIRQFLKKKIQEKGKRATIAW |
| | (SEQ ID NO: 9) |
| Syn71 | AGTEKIFQRLKKTIQEGKKIAKRWW |
| | (SEQ ID NO: 10) |

The confirmation and quantification of the MC1-2, Syn20, and Syn71 peptides chemisorbed onto the PhaNP surface was carried out using LC-MS analysis. The results on PhaNP@MC1-2 showed a residual peptide presence in primary and secondary nanoparticle supernatants of 15.78 μM and 1.4 μM, respectively. Therefore, the final concentration of the MC1-2 peptide on the PhaNPs was 51.57 μM. The results of PhaNP@Syn20 and PhaNP@Syn71 displayed no residual Syn20 or Syn71 peptide present in any of the supernatants, confirming that all of the peptides had been chemisorbed onto the PhaNP surface. The final concentrations of Syn20 and Syn71 on the PhaNPs were 49.44 μM and 91.92 μM, respectively.

Biocidal activity against five bacteria strains. PhaNP@Syn20 were tested against P. aeruginosa FRD1, S. aureus USA300, A. baumannii and K. pneumoniae, and PhaNP@Syn71 was tested on S. pyogenes. Two sizes of PhaNP@Syn20 and PhaNP@Syn71 were tested (20 nm and 95 nm) to study the effect of nanoparticle size at the same concentrations. 20 nm PhaNP@MC1-2 was tested against P. aeruginosa FRD1 and S. aureus USA300 to compare the difference in the bacteria inhibition between MC1-2 and Syn20-conjugated PhaNPs. For bacterial growth inhibition measurements, PhaNP@MC1-2 were concentrated three times three times concentrated (154.71 μM), and both PhaNP@Syn20 and PhaNP@Syn71 were concentrated to 150 μM.

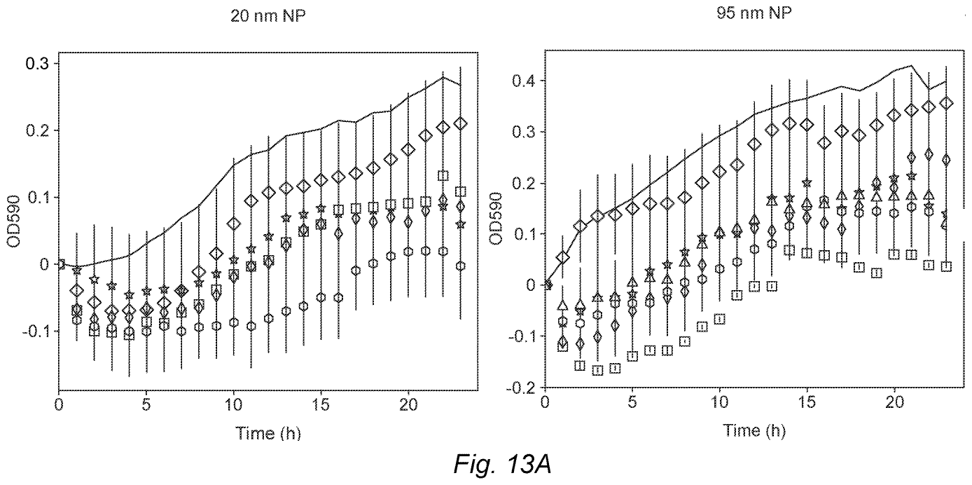

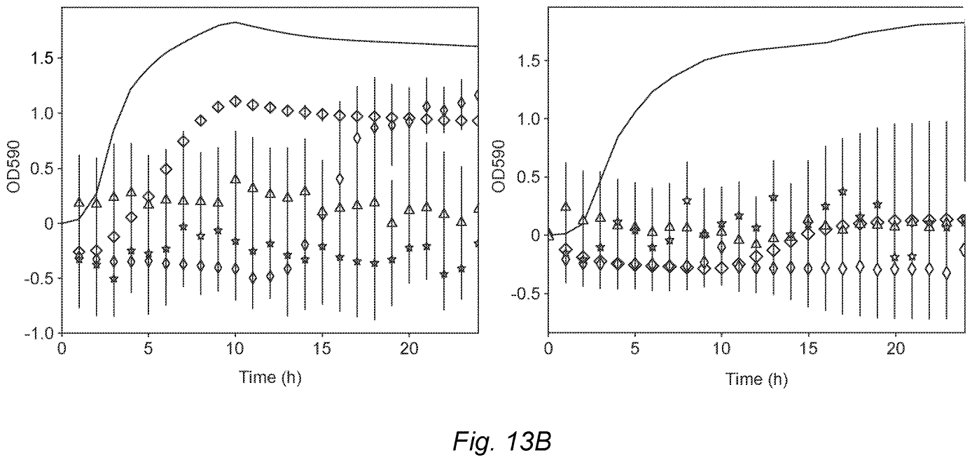

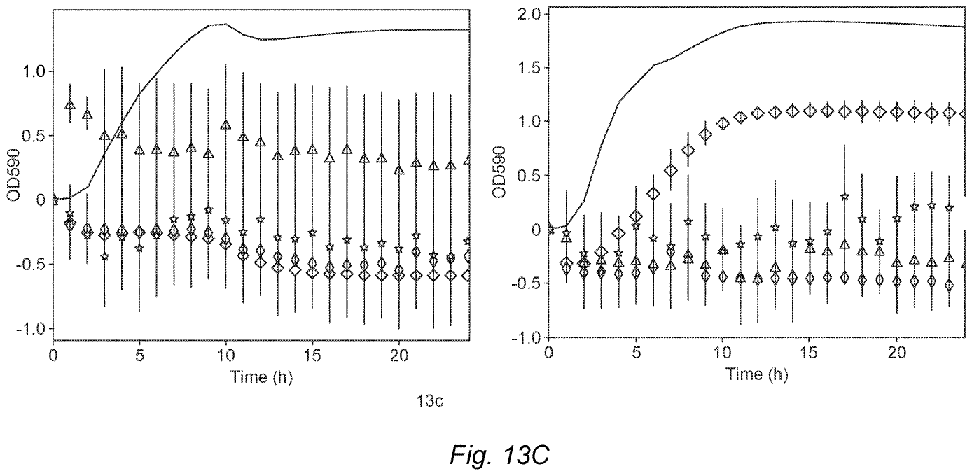

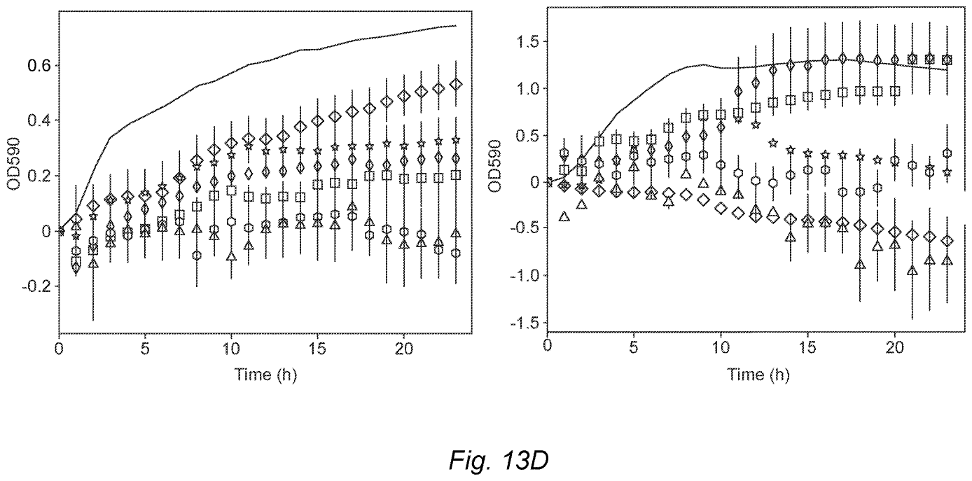

Pseudomonas aeruginosa FRD1. Pseudomonas aeruginosa FRD1 is a clinically relevant multi-drug resistant mucoid pathogen capable of forming biofilms, causing a diverse set of infections. P. aeruginosa FRD1 infections are especially relevant in causing chronic lung infections in cystic fibrosis patients, leading to poor patient recovery outcomes. The visualized results show growth inhibition of P. aeruginosa FRD1 for both 20 nm and 95 nm PhaNP@Syn20 (FIG. 13). Further analysis confirms the bacterial growth inhibition by PhaNP@Syn20 up to 100% in 20 nm nanoparticles and up to 90% in 95 nm nanoparticles (Table 5). 20 nm PhaNP@Syn20 inhibit P. aeruginosa FRD1 growth>55% at all concentrations tested (12.36-37.08 μM), with the highest concentration (37.08 μM) fully inhibiting the bacterial growth (100.99%). 95 nm PhaNP@Syn20 exhibit a 64.81% inhibition of the bacteria growth by the lowest concentration of PhaNP@Syn20 tested (12.36 μM), with the highest inhibition of 90.64% achieved with the concentration of 14.83 μM. The results indicate that overall, the smaller 20 nm PhaNP@Syn20 exhibit stronger antibacterial activity against P. aeruginosa FRD1 than 95 nm PhaNP@Syn20, reaching the highest antibacterial activity of 100.99±24.02% at a concentration of 37.08 μM, while the 95 nm particles exhibit maximum inhibition of 90.64±0.64% at a concentration of 14.83 μM. However, at the concentration of 14.83 μM, 20 nm PhaNP@Syn20 achieves only 59.11±32.91% inhibition. Therefore, it can be concluded that there are tradeoffs when using different-sized nanoparticles against P. aeruginosa FRD1 since an overall higher inhibition rate can be achieved by the smaller nanoparticles, while a lower concentration is needed for the bigger nanoparticles to achieve inhibition of >90%.

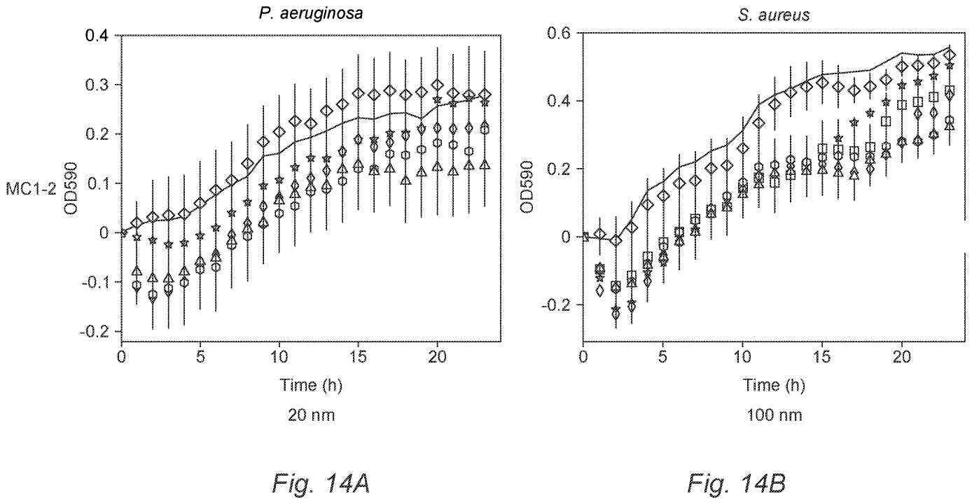

20 nm PhaNP@MC1-2 display an overall weak antibacterial activity against P. aeruginosa FRD1 (FIG. 14), with the highest inhibition rate reaching 49.82±29.90% at the highest concentration (51.56 μM) (Table 5). Lower concentrations (17.19 μM to 25.78 μM) exhibited bacterial inhibition of <25%. The results clearly indicate that the antibacterial activity of PhaNP@Peptide against P. aeruginosa FRD1 is depending on the AMP, with the PhaNP@Syn20 peptide being at least 3 times higher than PhaNP@MC1-2 at similar concentrations.

Staphylococcus aureus USA300. Staphylococcus aureus USA300 is a methicillin-resistant (MRSA) bacterial strain predominantly causing skin and soft tissue infections causing outbreaks in certain populations. PhaNP@Syn20 exhibit a significant inhibition of S. aureus USA300, both nanoparticle sizes fully inhibiting the bacterial growth (FIG. 13). 20 nm PhaNP@Syn20 inhibits S. aureus USA300 growth by 91.02±36.48% at 25 μM and by 111.18±14.64% at the highest 50 μM concentration (Table 5). The lowest PhaNP@Syn20 concentration (8 μM) only inhibits bacterial growth by 27.61±14.73%, 95 nm PhaNP@Syn20 exhibits a full growth inhibition already at 8 μM, displaying 106.86±11.13% inhibition rate, with higher concentrations (25-50 μM) inhibiting >90% of S. aureus USA300 growth. Therefore, the results clearly show that in the case of S. aureus USA300, bigger (95 nm) PhaNP@Syn20 exhibits significantly greater antibacterial activity at lower concentrations. In addition, 95 nm PhaNP@Syn20 displays an overall more stable inhibition fashion compared to the 25 nm nanoparticles.

The MC1-2-conjugated 20 nm PhaNPs display a weak antibacterial activity against S. aureus USA300 (FIG. 14), with the maximum inhibition reaching around 40% at the highest concentrations (25.78-51.56 μM) (Table 5). At lower concentrations of PhaNP@MC1-2, the inhibition stays <25%. Therefore, the results indicate clearly that the inhibition of S. aureus USA300 is dependent on the peptide conjugated onto the PhaNPs.

Acinetobacter baumannii. Multi-drug resistant Acinetobacter baumannii is one of the most troublesome pathogens in global healthcare, mainly found in hospital-acquired infection settings. A. baumannii strains resistant to all known antibiotics have been identified, making this bacteria one of the most frightening realities in the emerging antibiotic-resistance era. The bacterial growth inhibition measurements indicate that both 20 nm and 95 nm PhaNP@Syn20 inhibit A. baumannii growth up to 100% (FIG. 13). At the lowest concentration, both sizes (20 nm and 95 nm) of nanoparticles inhibit the bacterial growth by 133.30±23.30% and 117.16±1.30%, respectively (Table 5). However, the inhibition rate decreases to 76.28±13.87% for 20 nm PhaNP@Syn20 at the concentration of 25 μM, while for 95 nm PhaNP@Syn20 the inhibition stays >100%. Therefore, overall the results indicate that 95 nm PhaNP@Syn20 exhibits more stable inhibition of A. baumannii at all concentrations (8-50 μM) compared to 20 nm PhaNP@Syn20.

Klebsiella pneumoniae. Klebsiella pneumoniae is an opportunistic pathogen, becoming a concerning factor in healthcare settings due to its acquired antibiotic resistance that has led to many infections, such as urinary tract infections, pneumonia or bacteremia increasingly more challenging to treat. Both sizes (20 nm and 95 nm) of PhaNP@Syn20 exhibit antibacterial activity against K. pneumoniae up to 100% (FIG. 13). 20 nm PhaNP@Syn20 displays bacterial growth inhibition >55% at all concentrations (4-50 μM), with inhibition of >100% at concentrations from 25 μM (Table 5). 95 nm PhaNP@Syn20 exhibits inhibition of 90.37±2.83% at the lowest concentration of 4 μM, however, there is no inhibition observed in concentrations 8-10 μ.M. At the highest concentration (50 μM), 95 nm PhaNP@Syn20 displays inhibition of 169.26±37.82%. Overall, the results indicate that there are drawbacks to both nanoparticle sizes; 95 nm PhaNP@Syn20 exhibits an inhibition rate >90% already at 4 μM concentration, however, it loses its antibacterial activity at 8-10 μM. 20 nm PhaNP@Syn20, on the other hand, does not show high inhibition at the lowest concentration (4 μM), however, its inhibition stays relatively stable for all concentrations and reaches a full inhibition from 25 μM.

Streptococcus pyogenes. Streptococcus pyogenes (group A streptococcus) is a pathogen causing a wide range of infections ranging from mild strep throat to invasive streptococcal toxic shock syndrome. Although the bacteria is still somewhat susceptible to penicillin, the emergence of increasing antibiotic resistance has made it more and more difficult to treat S. pyogenes infections. PhaNP@Syn71 significantly inhibited the growth of S. pyogenes with both nanoparticle sizes (20 nm and 95 nm) (FIG. 14).

20 nm PhaNP@Syn71 exhibited the highest inhibition rate of 110.74±14.80% at concentration of 25 μM, while 95 nm PhaNP@Syn71 reached the highest inhibition of 104.38±10.71% (Table 5). At the concentration of >8 μM, 20 nm nanoparticles showed inhibition of >70%, 95 nm nanoparticles, however, display inhibition of <50% for concentrations 4-10 μM. Therefore, the results clearly indicate that smaller 20 nm PhaNP@Syn71 exhibits a greater antibacterial activity against S. pyogenes than 95 nm PhaNP@Syn71.

Size-dependence of the antibacterial activity. In the previous work on PhaNPs, it was shown that the antibacterial effect of the nanoparticles shows a size-dependent fashion. Many other works have studied and confirmed the size dependent antibacterial activity of various nanoparticles. Here, we further studied the effect of the PhaNP size on the antibacterial activity and show that the nanoparticle size plays an important role in its antibacterial activity level for some bacteria strains, while not being relevant for other strains. The results reported in previous sections indicate that there is a clear overall increase in the inhibition of various S. aureus USA300 and A. baumannii with larger (95 nm) PhaNP@Syn20 (Table 5). 95 nm PhaNP@Syn20 also showed a greater inhibition against K. pneumoniae at the lowest concentration of 4 μM, however, they lost the antibacterial activity for higher concentrations up to 10 μM and higher concentrations did not show an improved inhibition. P. aeruginosa FRD1 showed to be less responsive to our nanoparticle design and the chosen peptides. This tendency has been previously observed by other researchers studying the antibacterial nanoparticles. Lower susceptibility to the nanoparticles is likely due to more resistant lipid compounds on P. aeruginosa FRD1 outer membrane, making the interaction of the nanoparticles with the membrane more challenging.

The smaller 20 nm PhaNP@Syn20 displayed a greater inhibition than the 95 nm PhaNP@Syn20 against P. aeruginosa FRD1. In case of S. pyogenes, 20 nm PhaNP@Syn71 clearly exhibited a higher antibacterial activity. It has been previously confirmed that smaller particles exhibit higher antibacterial activity compared to bigger particles. Nanoparticle size is known to alter the chemical, physical and biological properties of the nanoparticles, leading to the difference in their activity against various bacteria strains. This study concludes that the size dependence is unique for each bacteria strain individually, and there is no uniform relationship between the nanoparticle size and their activity against all bacteria.

TABLE 5
Inhibition percentages and standard deviations of P. aeruginosa FRD1, S. aureus USA300,
A. baumannii, K. pneumoniae and S. pyogenes by PhaNPs conjugated with different
peptides (MC1-2, Syn20 and Syn71) and different sizes of nanoparticles (20 nm and 95 nm).
Inhibition (%)

| | | | 12.36 uM | 14.83 uM | 18.54 uM | 37.08 uM | 42.36 uM |
|---|---|---|---|---|---|---|---|
| Syn20 | 20 nm | P. aeruginosa FRD1 | 77.58 ± 39.23 | 59.11 ± 32.91 | 67.23 ± 24.02 | 100.99 ± 28.90 | NA |
| | 95 nm | | 64.81 ± 11.68 | 90.64 ± 0.64 | 38.62 ± 22.94 | 70.79 ± 20.15 | 68.60 ± 20.71 |
| | | | 8 uM | 25 uM | 50 uM | | |
| | 20 nm | S. aureus USA300 | 27.61 ± 14.73 | 91.02 ± 36.48 | 111.18 ± 14.64 | | |
| | 95 nm | | 106.86 ± 11.13 | 94.83 ± 23.85 | 92.18 ± 18.85 | | |
| | 20 nm | A. baumannii | 133.30 ± 23.30 | 76.28 ± 39.62 | 124.42 ± 41.96 | | |
| | 95 nm | | 117.16 ± 1.30 | 116.79 ± 24.71 | 98.47 ± 13.00 | | |
| | | | 4 uM | 8 uM | 10 uM | 25 uM | 50 uM |
| | 20 nm | K. pneumoniae | 55.51 ± 11.31 | 72.64 ± 13.87 | 64.67 ± 1.29 | 110.74 ± 14.80 | 100.74 ± 23.87 |
| | 95 nm | | 90.37 ± 2.83 | −9.28 ± 11.97 | −9.54 ± 29.80 | 74.36 ± 25.76 | 169.26 ± 37.82 |
| | | | 4 uM | 8 uM | 10 uM | 25 uM | 50 uM |
| Syn71 | 20 nm | S. pyogenes | 47.24 ± 14.98 | 69.85 ± 15.76 | 74.37 ± 30.08 | 102.52 ± 19.74 | 96.77 ± 18.40 |
| | 95 nm | | 19.05 ± 11.55 | 48.31 ± 38.35 | 45.49 ± 6.97 | 88.36 ± 10.63 | 104.38 ± 10.71 |
| | | | 10.31 uM | 17.19 uM | 20.63 uM | 25.78 uM | 51.56 uM |
| MC1-2 | 20 nm | P. aeruginosa FRD1 | NA | 4.94 ± 36.53 | 22.54 ± 29.93 | 25.27 ± 33.02 | 49.81 ± 29.90 |
| | | S. aureus USA300 | 9.29 ± 3.89 | 22.90 ± 10.55 | 25.67 ± 6.20 | 38.44 ± 6.55 | 40.99 ± 9.56 |

The effect of various peptides on PhaNP surface on the antibacterial activity. The results of various peptides chemisorbed onto the PhaNPs show a clear sign that certain peptides target the bacteria better. Comparing the MC1-2 and Syn20-conjugated PhaNPs, the results clearly indicate that Syn20-modified nanoparticles exhibit significantly higher antibacterial activity against P. aeruginosa FRD1 and S. aureus USA300 at the same concentrations. There are several potential reasons for Syn20 exhibiting better antibacterial activity than MC1-2. First, MC1-2 is a significantly smaller peptide (15 amino acids) than Syn20 (25 amino acids). Previous studies on antimicrobial peptides have shown that there are limits to the effective size reduction and the antimicrobial activity is reduced when the size of the peptide is smaller than the limit. In addition, MC1-2 is derived from an eukaryotic AMP while Syn20 is derived from a prokaryotic AMP. Therefore, we can hypothesize that prokaryotic AMPs exhibit higher antibacterial activity against bacteria strains, however, the reasons need to be studied further. It can be concluded that regardless of the Gram group, Syn20 exhibits higher inhibition against the bacteria than MC1-2, showing that bacteria-derived AMPs have greater antibacterial activity.

The results of bacteria growth curves in the presence of PhaNP@Peptide have confirmed that this design has the potential to be applied as an antibacterial agent. It has been established that there is a size dependence in the antibacterial activity of the nanoparticles against various bacteria strains, but it is unique for each bacteria strain, with no uniform relationship between the size and all bacteria strains. Regardless of the Gram group, some bacteria are more susceptible to smaller,

20 nm PhaNP@Peptide, while others are susceptible to 95 nm PhaNP@Peptide. A peptide dependence was observed, displaying that Syn20-modified PhaNPs display significantly greater inhibition than MC1-2-modified nanoparticles. No significant difference in the antibacterial activity of the nanoparticles against gram-positive (S. aureus USA300 and S. pyogenes) and gram-negative (A. baumannii and K. pneumoniae) bacteria were observed, indicating the universality of our PhaNP@Peptide design to be used against various bacteria strains.

### Example 4. Treatments to Reduce Bacterial Load in Wounds

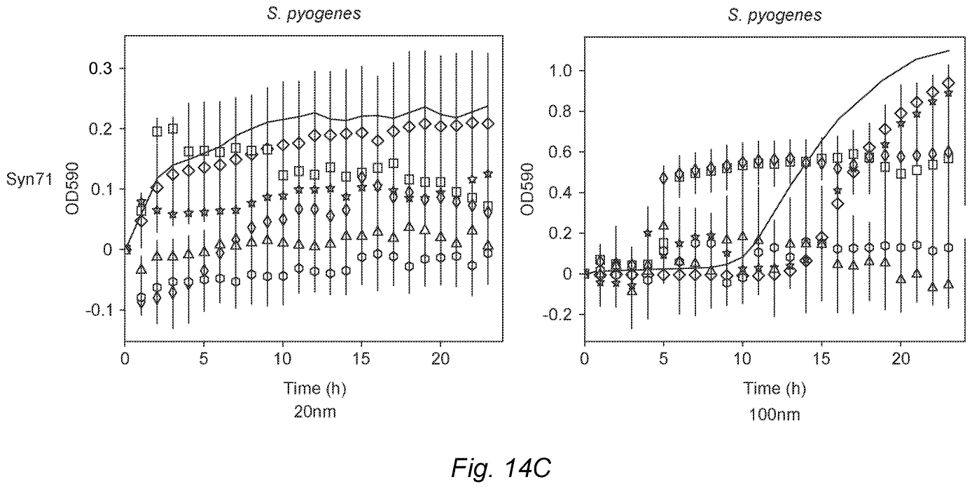

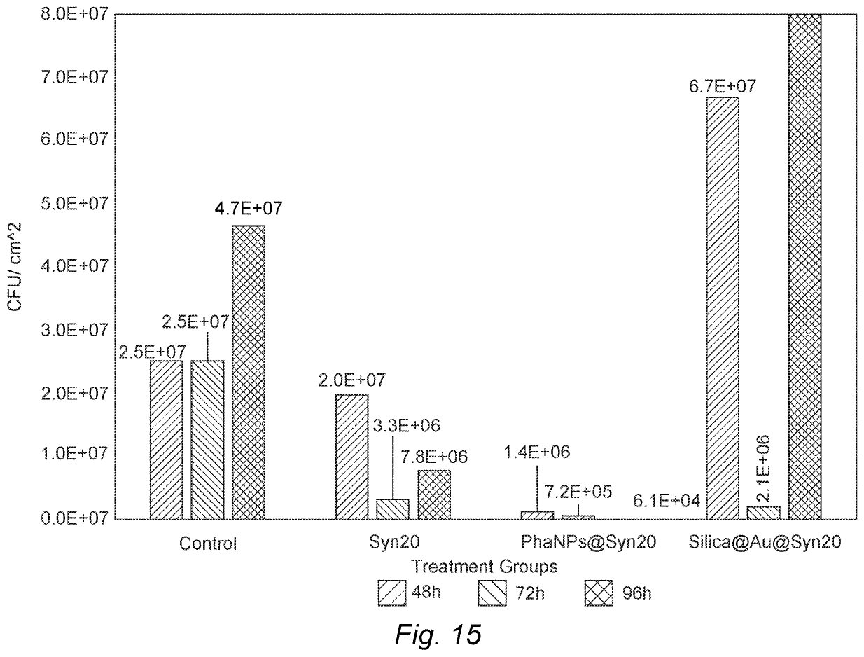

FIG. 15 shows a single daily topical dose of PhANPs-Peptide 24 h post bacterial infection for treating 10⁸ CFU of MRSA, or MDR A. baumannii applied to biopsy punched induced wounds on Sprague Dawley rats, to create wound infection models. Three cohorts. Cohort 1=1 dose PhANPs 24 h post bacteria exposure; Cohort 2=same as cohort 1 but 2 doses, 24 h apart; Cohort 3=same as cohort1 but 3 doses and each dose was 24 h apart. Cohort 1 was sacked 48 h post-bacterial infection, Cohort 2 was sacked 72 h post-bacterial infection, and Cohort3 was sacked 96 h post-bacterial infection. The PhANPs-peptide reduced bacterial load by 100-fold to 1000-fold in comparison to the no treatment groups. The PhANPs-peptide showed a 10-fold decrease in methicillin resistant S. aureus USA300 CFU counts in the wound after 1 application, 100-fold decrease after 2 applications, and 1000-fold decrease after 3-applications. Each dose application was 24 hours apart. After the application of the third dose the bacterial CFU load fell below infection threshold levels.

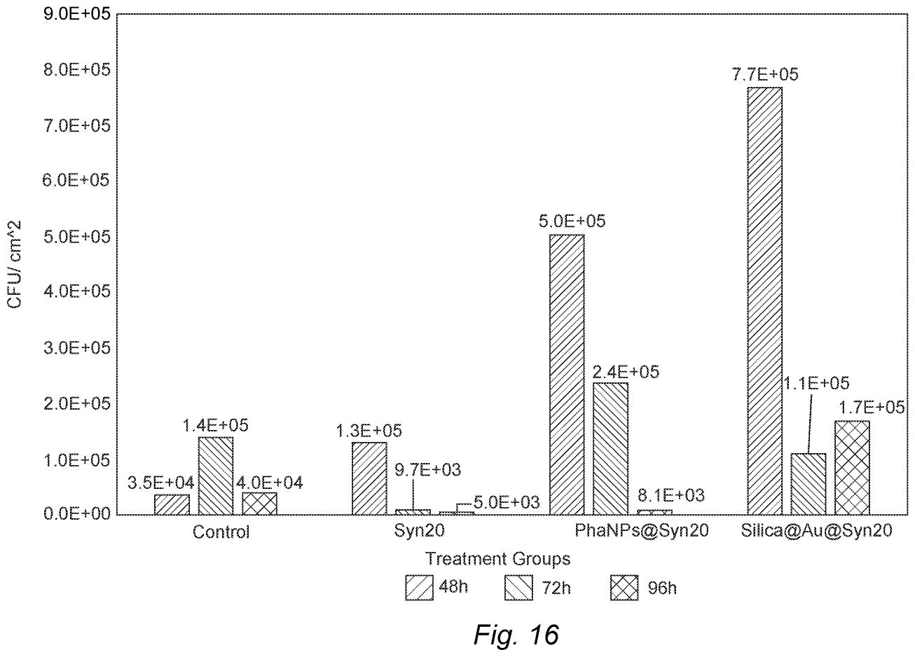

FIG. 16 shows single daily topical dose of PhANPs-Peptide 24 h post bacterial infection. Three cohorts. Cohort 1=1 dose PhANPs 24 h post bacteria exposure; Cohort 2=same as cohort 1 but 2 doses, 24 h apart; Cohort 3=same as cohort 1 but 3 doses and each dose was 24 h apart. Cohort 1 was sacked 48 h post-bacterial infection, Cohort 2 was sacked 72 h post-bacterial infection, and Cohort3 was sacked 96 h post-bacterial infection. The PhANPs-peptide reduced bacterial load by 100-fold to 1000-fold in comparison to the no treatment groups. The PhANPs-peptide showed a 100-fold decrease in Carbapenem Resistant A. baumannii BA1605 CFU counts in the wound after 3 applications. The CFU count did not increase once the PhANPs-peptide dose application started. Each dose application was 24 hours apart. After the application of the third dose the bacterial CFU load fell well below 10⁴ CFU and was well below infection threshold levels.

The PhANPs-Peptide consistently reduced bacterial load at wound site with just one dose applied every 24 h. It is possible to apply multiple doses in a 24 h period due to high biocompatibility. The antibacterial nanoparticles used in these experiments included the Syn20 peptide chemisorbed to nanoparticle surface at a concentration of 150 μM and 3% w/v silica in the composition.

### Example 5. Evolution of Resistance Studies Against Phage-Mimicking Nanoparticles

| | | | MIC | MIC | | |
|---|---|---|---|---|---|---|
| | | | after 28 | after 28 | MIC after | |
| | | | days | days | 28 days | |
| | | MIC of | exposure | exposure | exposure | |
| | | Ancestral | to | to | to | |
| | | Strain | 0.1 × MIC | 0.2 × MIC | 0.35 × MIC | |
| | | (“AS”) | of AS | of AS | of AS | |
| Bacteria | NP size | (mM) | (mM) | (mM) | (mM) | Resistance |
| S. aureus | 25 nm | 6.766 | 6.671 | 5.394 | 5.807 | none at any |
| USA300 | PhANPS@Syn20 | | | | | concentrations |
| | | | | | | tested |
| | 100 nm | 8.869 | 5.463 | 4.96 | 7.704 | none at any |
| | PhANPS@Syn20 | | | | | concentrations |
| | | | | | | tested |
| A. | 25 nm | 5.527 | 8.049 | 9.658 | 9.637 | slight |
| baumannii | PhANPS@Syn20 | | | | | emergence of |
| BA1605 | | | | | | resistance up |
| | | | | | | to 0.35 × MIC |
| | 100 nm | 7.6 | 7.012 | 6.102 | 5.019 | none at any |
| | PhANPS@Syn20 | | | | | concentrations |
| | | | | | | tested |
| K. | 25 nm | 6.053 | 5.564 | 5.895 | 9.636 | none at any |
| pneumoniae | PhANPS@Syn20 | | | | | concentrations |
| | | | | | | tested |
| | 100 nm | 5.341 | 11.608 | 6.049 | 5.774 | none from |
| | PhANPS@Syn20 | | | | | 0.2 × MIC |
| S. pyogenes | 25 nm | 5.596 | 18.038 | 9.931 | 5.525 | none at |
| | PhANPS@Syn71 | | | | | 0.35 × MIC |
| | 100 nm | 6.875 | 26.919 | 9.704 | 7.173 | none at |
| | PhANPS@Syn71 | | | | | 0.35 × MIC |
| P. | 25 nm | 6.88 | 8.299 | 7.015 | | none from |
| aeruginosa | PhANPS@Syn20 | | | | | 0.2 × MIC |
| FRD1 | | | | | | |
| | 100 nm | 7.019 | 14.16 | 5.656 | 7.025 | none from |
| | PhANPS@Syn20 | | | | | 0.2 × MIC |
| C. striatum | 25 nm | 7.782 | 6.918 | 6.018 | No | none |
| | PhANPS@Syn20 | | | | growth | from0.5 × |
| | | | | | | MIC |
| | 100 nm | 4.64 | 37.484 | 10.532 | No | Emergence of |
| | PhANPS@Syn20 | | | | growth | resistance at |
| | | | | | | sub-MIC |
| E. faecalis | 25 nm | 6.846 | 6.636 | 9.4 | No | Slight |
| | PhANPS@Syn20 | | | | growth | emergence of |
| | | | | | | resistance at |
| | | | | | | 0.9 × MIC |
| | 100 nm | 5.191 | 8.086 | 6.0194 | No | none at |
| | PhANPS@Syn20 | | | | growth | 0.9 × MIC |

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, in particular, U.S. Patent Publication No. 2021/0252162 to Nallathamby et al. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. An antibacterial nanoparticle comprising:
an inner silica core;
a gold (Au) nanosphere surrounding the inner silica core;
a surface layer comprising silver (Ag) surrounding the gold nanosphere or the silver is alloyed into the gold nanosphere; and
an antimicrobial peptide conjugated to the surface layer, wherein the antimicrobial peptide is selected from the group consisting of a cysteine terminated Syn71 peptide, a cysteine terminated MC1-2 peptide, and a cysteine terminated Syn20 peptide.

2. The antibacterial nanoparticle of claim 1, wherein the antibacterial peptide is the cysteine terminated Syn71 peptide, wherein the cysteine terminated Syn71 peptide comprises an amino acid sequence of CAGTEKIFQRLKKTIQEGKKIAKRWW (SEQ ID NO:1).

3. The antibacterial nanoparticle of claim 1, wherein the antibacterial peptide is the cysteine terminated MC1-2 peptide, wherein the cysteine terminated MC1-2 peptide comprises an amino acid sequence of CSAVWRHWRRFWLRKRK (SEQ ID NO: 2).

4. The antibacterial nanoparticle of claim 1, wherein the antibacterial peptide is the cysteine terminated Syn20 peptide, wherein the cysteine terminated Syn20 peptide comprises an amino acid sequence of CAWKKTIRQYLKNKIKKKGRKAVIAW (SEQ ID NO: 3).

5. The antibacterial nanoparticle of claim 1, wherein the inner silica core has a silanized outer surface.

6. The antibacterial nanoparticle of claim 1, wherein the nanoparticle has an average diameter of about 15 nm to about 30 nm.

7. The antibacterial nanoparticle of claim 1, wherein the nanoparticle has an average diameter of about 90 nm to about 100 nm.

8. The antibacterial nanoparticle of claim 1, wherein:
a Syn71 peptide is conjugated to the surface layer at an average concentration of about 40 μM to about 60 μM;
an MC1-2 peptide is conjugated to the surface layer at an average concentration of about 40 μM to about 60 μM; or
a Syn20 peptide is conjugated to the surface layer at an average concentration of about 40 μM to about 60 μM.

9. An antibacterial composition comprising the antibacterial nanoparticle of claim 1 and pharmaceutically acceptable diluent, excipient, or carrier.

10. The antibacterial composition of claim 9, wherein the composition comprises a population of the antibacterial nanoparticle having an average of from about 1 μM to about 150 μM of the antimicrobial peptide conjugated to the surface layer.

11. The antibacterial composition of claim 10, wherein the antibacterial composition is formulated as a topical cream or ointment.

12. A method of treating a bacterial infection in a subject having a bacterial infection comprising administering an effective amount of the antibacterial nanoparticles according to claim 1 to the subject, wherein the antibacterial nanoparticles reduce growth of the bacterial infection by greater than about 95%, kills bacteria of the bacterial infection, or both, thereby treating the bacterial infection.

13. The method of claim 12, wherein the bacterial cell is one or more of Enterococcus faecalis, Corynebacterium striatum, Staphylococcus aureus, Klebsiella pneumoniae, Acinetobacter baumannii, Pseudomonas aeruginosa, and Streptococcus pyogenes.

14. The method of claim 12, wherein antibacterial nanoparticle is administered topically or intravenously.

15. A medical bandage comprising the antibacterial nanoparticle of claim 1.

16. A method of treating a wound comprising applying the medical bandage of claim 15 to the wound.

17. A medical product comprising the antibacterial nanoparticle of claim 1, wherein the medical product is one or more of a medical implant, a dental implant, and a surgical instrument.

18. The medical product of claim 17, wherein the antibacterial nanoparticle is disposed on a surface of the one or more of the medical implant, the dental implant, and the surgical instrument.
